SEQUENCE LISTING (7 sequences)

SEQ ID NO: 1 - protein, 180 aa, Homo sapiens
  PEPTIDE (1)..(180): Human Leukemia inhibitory factor (LIF)
SPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 2 - DNA, 26 bp, Artificial Sequence
  Primer
caaaccgttgtgtgatttgttaatta

SEQ ID NO: 3 - DNA, 23 bp, Artificial Sequence
  Primer
gctttttgtccagatgtctttgc

SEQ ID NO: 4 - DNA, 36 bp, Artificial Sequence
  primer
tgtattaactgtcaaaagccactgtgtcctgaagaa

SEQ ID NO: 5 - DNA, 21 bp, Artificial Sequence
  primer
gtgactctacgcttcggttgt

SEQ ID NO: 6 - DNA, 20 bp, Artificial Sequence
  primer
gcccattaacaggtgttcca

SEQ ID NO: 7 - DNA, 30 bp, Artificial Sequence
  primer
cgtacaaagcacacacgtagacattcgtac

(19) United States
(12) Patent Application Publication
(10) Pub. No.: US 2012/0020914 A1
(43) Pub. Date: Jan. 26, 2012

(54) LEUKEMIA INHIBITORY FACTOR (LIF) FOR USE IN REPRESSING HUMAN PAPILLOMAVIRUS (HPV) TRANSCRIPTION

(75) Inventors: Joseph M. Bay, Palo Alto, CA (US); Bruce K. Patterson, Palo Alto, CA (US); Nelson Teng, Hillsborough, CA (US)

(73) Assignee: THE BOARD OF TRUSTEES OF THE LELAND STANFORD JUNIOR UNVERSITY, PALO ALTO, CA (US)

(21) Appl. No.: 13/167,404

(22) Filed: Jun. 23, 2011

Related U.S. Application Data
(60) Provisional application No. 61/398,397, filed on Jun. 23, 2010.

(51) Int. Cl. A61K 38/19; A61P 15/00; A61P 35/00; A61P 31/20

(52) U.S. Cl. 424/85.1

(57) ABSTRACT

Embodiments of the invention are related to leukemia inhibitory factor (LIF) for use in repressing human papillomavirus (HPV) transcription. Processes and related kits are described for treating a HPV-associated papillomatous proliferation, for treating a HPV-associated genital, anal, vulvar, penile, oral, or laryngeal wart, for treating HPV-associated cervical dysplasia or cervical cancer, and for repressing HPV transcription, by administering LIF to a patient in need thereof. A related embodiment is treatment of HPV-16 by use of LIF.

### RELATED APPLICATION

This application claims priority and other benefits from U.S. Provisional Patent Application Ser. No. 61/398,397, filed Jun. 23, 2010, entitled “Leukemia inhibitory factor (LIF) for use in repressing human papillomavirus (HPV) transcription”. Its entire content is specifically incorporated herein by reference.

### TECHNICAL FIELD OF THE INVENTION

The present invention relates to the field of biological compositions and their use in repressing viral transcription, particularly, in repressing human papillomavirus transcription.

### BACKGROUND

Human Papillomavirus (HPV) causes cervical cancer, the second largest cause of cancer mortality in women worldwide. The high-risk HPV types 16 and 18 account together for as many as 70% of cervical cancers. Two genes, in particular, that are encoded by HPV, namely E6 and E7, interact with important cellular systems and are believed to contribute to a substantial degree to a cell's oncogenic transformation, which ultimately leads to cancer. The degree to which these genes are transcribed in infected cells is a good predictor of cancer progression, since high expression of the E6 and E7 mRNA transcripts directly correlate with cervical cancer progression.

It would be highly desirable to have methods available to target and interfere with the expression of E6 and E7 to halt cervical cancer progression.

### SUMMARY

The present invention teaches methods related to leukemia inhibitory factor (LIF) for repressing human papillomavirus (HPV) transcription to halt cervical cancer progression.

A first embodiment is a process for treating a HPV-associated papillomatous proliferation in a patient comprising: administering LIF or polypeptide, said polypeptide being at least 30% identical thereto or with up to 30% insertions, deletions, or conservative substitutions therein, topically to a HPV-associated papillomatous proliferation in a patient in need thereof.

A second embodiment is a process for treating a HPV-associated genital, anal, vulvar, penile, oral, or laryngeal wart in a patient comprising: administering LIF or polypeptide, said polypeptide being at least 30% identical thereto or with up to 30% insertions, deletions, or conservative substitutions therein, topically to a HPV associated genital, anal, vulvar, penile, oral, or laryngeal wart in a patient in need thereof.

A third embodiment is a process for treating HPV-associated cervical dysplasia or cervical cancer in a patient comprising: identifying a patient in need thereof, and administering LIF or polypeptide, said polypeptide being at least 30% identical thereto or with up to 30% insertions, deletions, or conservative substitutions therein, to said patient.

A fourth embodiment is a process for repressing HPV transcription in a patient comprising: detecting HPV DNA or RNA in a cervical swab or biopsy sample of a patient, and administering LIF or polypeptide, said polypeptide being at least 30% identical thereto or with up to 30% insertions, deletions, or conservative substitutions therein, to said patient.

A fifth embodiment is a kit comprising: purified or recombinant LIF or polypeptide, said polypeptide being at least 30% identical thereto or with up to 30% insertions, deletions, or conservative substitutions therein, and a component configured to collect a cervical swab or biopsy sample to test for HPV.

A sixth embodiment is the process of the first or second embodiment further comprising as a first step identifying a patient in need thereof.

A seventh embodiment is the process or kit of any of the first to fifth embodiment, wherein said HPV is HPV-16, HPV-18, HPV-31, or HPV-45, or HPV-33, HPV-35, HPV-52, or HPV-58.

An eighth embodiment is the process or kit of any of the first to fifth embodiment, wherein said HPV is HPV-16, HPV-18, HPV-31, or HPV-45.

A ninth embodiment is the process or kit of any of the first to fifth embodiment, wherein said HPV is HPV-16.

A tenth embodiment is the process of the second embodiment, wherein said HPV-associated wart is a genital wart.

An eleventh embodiment is the process of the third embodiment, wherein said patient is diagnosed with HPV-associated cervical dysplasia classified as CIN1, CIN2, or CIN3.

A twelfth embodiment is the process of any of the first to fourth embodiment, wherein said patient is a human.

A thirteenth embodiment is the process of any of the first to fourth embodiment, wherein said patient is a female human.

A fourteenth embodiment is the process of any of the first to fourth embodiment, wherein said LIF or polypeptide is applied topically as an ointment, a transdermal drug delivery system, suppository, poultice, paste, powder, dressing, cream, or plaster.

A fifteen embodiment is the process of any of the first to fourth embodiment, wherein said administering step is by topical intravaginal application.

A sixteenth embodiment is the process or kit of any of the first to fifth embodiment, wherein said polypeptide comprises an amino acid sequence that is at least 70%, 75%, 80%, 85%, 90%, or 95% identical to SEQ ID NO:1.

A seventeenth embodiment is the process or kit of any of the first to fifth embodiment, wherein said polypeptide comprises the amino acid sequence of SEQ ID NO:1, but with up to 30%, 25%, 20%, 15%, 10%, or 5% insertions, deletions, or conservative substitutions.

An eighteenth embodiment is the process or kit of the sixteenth or seventeenth embodiment, wherein said polypeptide represses LCR activity in SiHa, represses transcription of E6, E7 mRNA or inhibits CaSki proliferation in culture.

A nineteenth embodiment is the process or kit of any of the first to fifth embodiment, wherein said LIF or polypeptide comprises the amino acid sequence of SEQ ID NO:1.

A twentieth embodiment is the process or kit of any of the first to fifth embodiment, wherein said LIF or polypeptide consists of the amino acid sequence of SEQ ID NO:1.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates a phylogenetic tree containing the sequences of 118 Papillomavirus Types (De Villiers et al., 2004).

FIG. 2 illustrates the organization of the HPV Genome (Doorbar, 2006).

FIG. 3 illustrates the inhibitory effect of LIF on HPV-16 Long Control Region (LCR)-driven transcription. Panel A: Luciferase expression in LIF-treated and untreated SiHa pGLuc cells. Panel B: CaSki cells were treated with the indicated concentrations of LIF for 24 hours. Quantitative real-time PCR was performed, as described. Error bars represent standard error of the mean. Panel C: CaSki and SiHa cells were treated with the indicated concentrations of LIF for 72 hours. Quantitation of E6 relative to β-actin is represented in arbitrary units. Error bars represent standard error of the mean. Asterisks represent significance (single asterisk for P<0.1, double asterisk for P<0.05).

FIG. 4 illustrates that LIF activates STAT3. Panel A. SiHa cells were stimulated with LIF (10 ng/mL) for the times indicated (in minutes), and the level of phospho-STAT3 (y705) measured. STAT3 is transiently phosphorylated following stimulation with LIF within 60 minutes, returning to baseline level by 120 minutes. Histograms are colored according to the log₁₀-fold increase in mean fluorescence intensity relative to unstimulated cells. Panel B. CaSki cells transfected with a STAT3 reporter plasmid were treated with 50 ng/mL LIF for 6 hours prior to assay. The increase in relative light units is shown. Error bars represent standard error of the mean. Asterisks represent significance (single asterisk for P<0.1, double asterisk for P<0.05).

FIG. 5 illustrates the proliferation of HPV-transformed cells. CaSki cells grown for 40 hours in the presence of the indicated agents were assayed by MTT for proliferation/metabolic activity. EGF and IL-6 enhanced cell number, while LIF inhibited proliferation. Error bars represent standard error of the mean. Asterisks represent significance (single asterisk for P<0.1, double asterisk for P<0.05).

### DEFINITIONS

The practice of the present invention may employ conventional techniques of chemistry, molecular biology, recombinant DNA, genetics, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

### DETAILED DESCRIPTION

The present invention teaches methods related to leukemia inhibitory factor (LIF) for repressing human papillomavirus (HPV) transcription to halt cervical cancer progression.

### Classification of Papillomaviruses

Papillomavirus (PV) isolates are traditionally described as “types”. PVs cause benign tumors (warts, papillomas) in their natural host and occasionally in related species. Papillomas are induced in the skin and mucosal epithelia, often at specific sites of the body. Some papillomatous proliferations induced by specific types of PVs bear a high risk for malignant progression. PVs have circular double-stranded DNA genomes with sizes close to 8 kb. In spite of their small size, their molecular biology is complex. Three oncogenes, E5, E6, and E7, modulate the transformation process, two regulatory proteins, E1 and E2, modulate transcription and replication, and two structural proteins, L1 and L2, compose the viral capsid. Most cis-responsive elements are in the long control region (LCR) between L1 and E6. The papilloma-viruses had been originally lumped together with the polyomaviruses in one family, the Papovaviridae. This was based on similar, nonenveloped capsids and the common circular double-stranded DNA genomes. As it was later recognized that the two virus groups have different genome sizes, different genome organizations, and no major nucleotide or amino acid sequence similarities, they are now officially recognized by the International Committee on the Taxonomy of Viruses (ICTV) as two separate families, Papillomaviridae and Polyomaviridae.

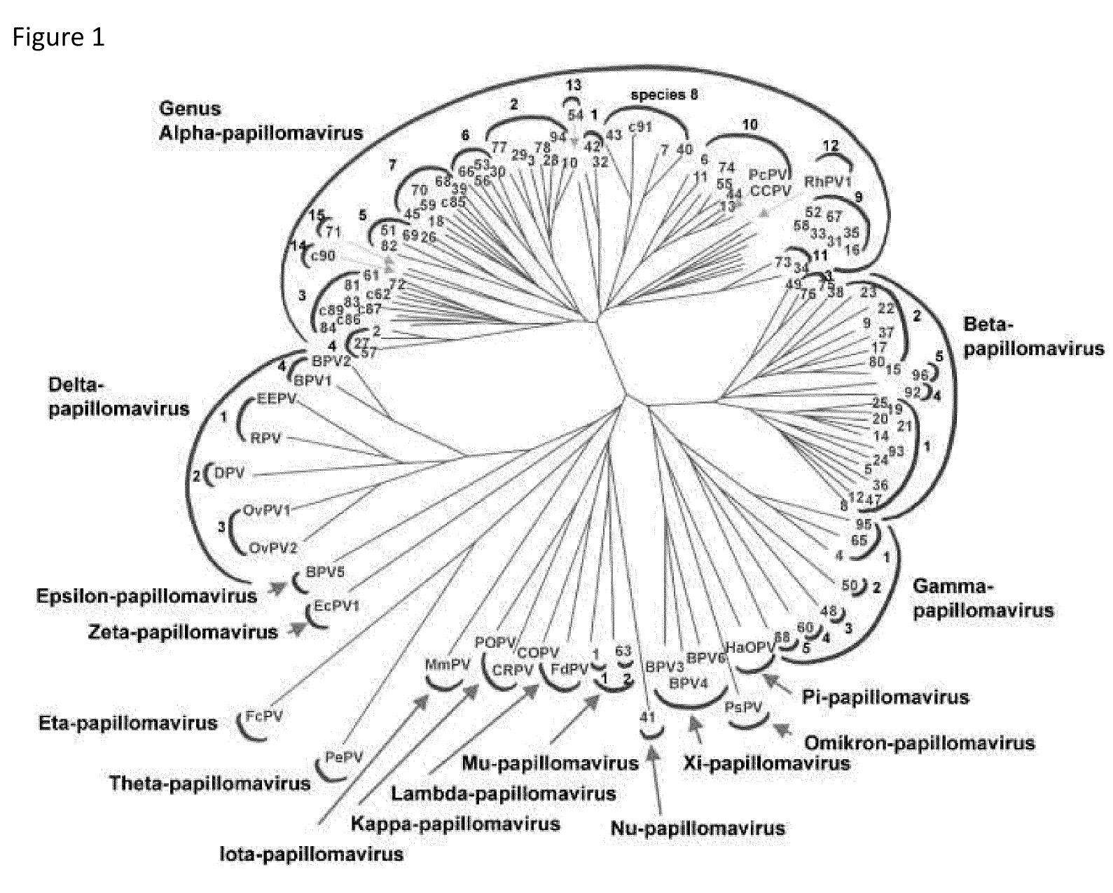

The L1 ORF is the most conserved gene within the genome and has therefore been used for the identification of new PV types over the past 15 years. A new PV isolate is recognized as such if the complete genome has been cloned and the DNA sequence of the L1 ORF differs by more than 10% from the closest known PV type. Differences between 2% and 10% homology define a subtype and less than 2% a variant. A cladogram based on the complete L1 ORF of 96 HPV types and 22 animal papillomavirus types is presented in FIG. 1. The frequency distribution of pairwise identity percentages from sequence comparisons of the L1 ORF demonstrates three taxonomic levels, both when complete genomes are compared, and based on the comparison of L1 genes, namely genera, species, and types. These observations of deVilliers (deVilliers et al., 2004) were integrated with the classification standards established by ICTV and led to an official interpretation of phylogenetic clusters of PV types as “genera” and “species”, respectively. FIG. 1 illustrates the phylogenetic tree containing the sequences of 118 papillomavirus types, based on L1 ORF sequences. The numbers at the ends of each of the branches identify an HPV type; c-numbers refer to candidate HPV types. All other abbreviations refer to animal papillomavirus types. The outermost semicircular symbols identify papilloma-virus genera, e.g., the genus alphapapillomavirus. The number at the inner semicircular symbol refers to papillomavirus species. To give an example, the HPV types 7, 40, 43, and c91 together form the HPV species 8 in the genus alpha-papillomavirus.

Higher-order clusters of HPV types are termed “genus”. Different genera share less than 60% nucleotide sequence identity in the L1 ORF. Conversely, within a given genus, the L1 DNA of all members share more than 60% identity. Lower-order clusters of HPV types are termed “species”. Such species within a genus share between 60% and 70% nucleotide identity. The traditional PV types within a species share between 71% and 89% nucleotide identity within the complete L1 ORF.

Using this classification, HPV are clustered among 5 of the 12 genera: alpha, beta, gamma, mu, and nu, with the other 7 genera being comprised exclusively of animal PV. The HPV of greatest medical importance (i.e., those that are associated with genital and mucosal cancers) are members of the alpha genus. Most alpha PVs primarily infect genital and nongenital mucosal surfaces and the external genitalia. This group of PV is often referred to collectively as the genital-mucosa types. The types that are associated with cervical cancer, often designated as high risk types, are found in species 5, 6, 7, 9, and 11. HPV-16, the type found most frequently in cervical cancer, is a member of species 9, whereas the next most common cancer-associated 10 type, HPV-18, is a member of species 7. HPV6 causes most cutaneous genital warts, followed by HPV 11, with both being members of species 10. In contrast to most species of the alpha genus, members of alpha species 4 (HPV2, 27, and 57) are primarily infectious for nongenital skin. The beta, gamma, mu, and nu viruses also infect nongenital skin.

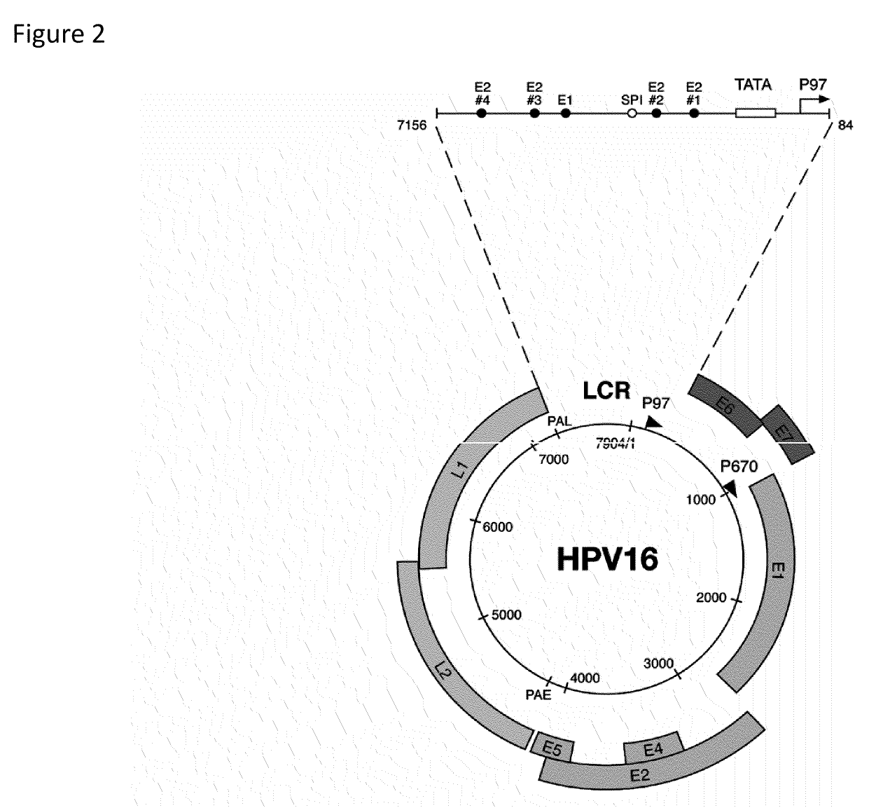

All papillomaviruses share a number of characteristics and contain doublestranded circular DNA within an icosahedral capsid (FIG. 2). In FIG. 2, the HPV16 genome (7904 bp) is shown as a black circle with the early (p97) and late (p670) promoters marked by arrows. The six early ORFs (open reading frames), E1, E2, E4 and E5 (on the one hand) and E6 and E7 (on the other hand), are expressed from either p97 or p670 at different stages during epithelial cell differentiation. The late ORFs, L1 and L2, are also expressed from p670, following a change in splicing patterns, and a shift in polyadenylation site usage. All the viral genes are encoded on one strand of the double-stranded circular DNA genome. The long control region (LCR from 7156-7184) is enlarged to allow visualization of the E2-binding sites and the TATA element of the p97 promoter. The location of the E1- and SP1-binding sites is also shown. SP1 is a transcription factor. E2 is a virally encoded regulatory protein. E1 is a virally encoded replication factor.

The key events that occur following infection are as follows. Infection leads to the establishment of the viral genome as a stable episome (without integration into the host cell genome) in cells of the basal layer. In the basal cells, it appears that the viral genome replicates with the cellular DNA during S-phase, with the replicated genomes being partitioned equally during cell division. Viral episomes may be maintained at 10-200 copies in basal cells. The appearance of cells in the epidermis expressing cell cycle markers above the basal layer is a consequence of virus infection, and in particular, the expression of the viral oncogenes, E6 and E7. The expression of viral proteins necessary for genome replication occurs in cells expressing E6 and E7 following activation of p670 in the upper epithelial layers. The L1 and L2 genes are expressed in a subset of the cells that contain amplified viral DNA in the upper epithelial layers. Cells containing infectious particles are eventually shed from the epithelial surface. In cutaneous tissue, this follows nuclear degeneration and the formation of flattened squames. The expression of E6 and E7 in the presence of low levels of E1, E2, E4 and E5 allows maintenance of the viral genome. Elevation in the levels of these replication proteins facilitates viral genome amplification. The first appearance of L2 allows genome packaging to begin, with the expression of L1 allowing the formation of infectious virions and virus release.

### Papillomaviruses and Cancer

Whereas some PV do not appear to have oncogenic potential, a subset of PV is clearly implicated in the development of malignancy in humans and animals. In humans, these include several mucosal epithelial cancers. Cervical cancer is a particularly relevant form of cancer from a public health perspective. HPV are also implicated in other anogenital cancers, including anal cancer, vulvar cancer, and penile cancer, as well as in oral and laryngeal cancers.

Cervical cancer is the second most common malignancy among women worldwide. Despite its worldwide distribution, the frequency of cervical cancer varies considerably, being about ten times more common in some countries than in others. About 80% of cervical cancer occur in developing countries. It occurs less frequently in developed countries. In the United States, approximately 12,000 new cases are diagnosed annually, and about one third of these women will die of their malignant disease. Most cancers occur in the transformation zone of the cervix, where the columnar cells of the endocervix form a junction with the stratified squamous epithelium of the exocervix. About 85% of cervical cancers are squamous cell cancers. Most of the other cases are adenocarcinomas, with a small number being other tumors. Lesions that are destined to become malignant squamous cell carcinomas typically undergo a series of dysplastic changes over a time span of many years. The severity of the lesion is determined by the degree to which the squamous epithelium is replaced by basaloid cells, with the entire thickness being replaced in the most severe dysplasias. In the histologic classification of cervical intraepithelial dysplasia (CIN), grades 1, 2, and 3 correspond, respectively, to mild dysplasia, moderate dysplasia, and severe dysplasia or carcinoma in situ. The cervical dysplasias have their counterpart in the exfoliated cells present in the Papanicolaou (Pap) smear, by the presence of basaloid cells and koilocytosis.

Most dysplasias do not progress and, in fact, resolve spontaneously, with the likelihood of resolution decreasing with the severity of the dysplasia. More severe dysplasias, however, generally arise from less dysplastic lesions after several years, although a proportion of high-grade dysplasias can develop rapidly without passing through a low-grade stage. Because of the long interval between the development of cervical dysplasia and the development of invasive cancer, Pap smear screening programs can identify most premalignant lesions. Appropriate follow-up of women with these abnormalities, together with appropriate treatment, can thereby prevent the development of many cases of cervical cancer.

Today it is very well established that infection with specific types of HPV can cause cervical cancer. HPV types that are found more frequently in cervical cancers than in controls are designated as high risk. Otherwise, HPV types that are found less frequently in tumors than in controls are designated low risk. The high-risk HPV types, HPV-16, HVP-18, HPV-31, and HPV-45, account for close to 80% of the HPV-positive cancers. Other high-risk HPV include HPV-33, HPV-35, HPV-52, and HPV-58. Conversely, low-risk HPV types, like HPV-6 and HPV-11, are found infrequently in these cancers. HPV-16 is the most oncogenic HPV type, with HPV-18 the next most virulent. Together, these two types account for about 70% of cervical cancer. When asymptomatic, prevalent HPV infection was followed prospectively over a 10-year period, HPV-16 and HPV-18 were found to be substantially more likely to progress to CIN3 or invasive cancer compared with other HPV types, with the rate of progression being higher for HPV-16 than for HPV-18.

PV produce a chronic infection of stratified squamous epithelia of cutaneous or mucosal surfaces. To establish this infection, it is believed that the virus must infect epithelial cells that possess long-term proliferative capacity located in the basal cell layer of the epithelium. In an established infection, the lower layers of the epidermis harbor low numbers of episomal viral genomes and transcribe low levels of viral RNA. Vegetative viral DNA replication, high-level expression of the viral proteins, and virus assembly all occur in the upper epidermal layers, which are undergoing terminal differentiation. Virus assembly occurs in the nucleus, where virions often remain until after the cells are desquamated into the environment.

Genital HPV can infect the genital skin, the vaginal tract, or the cervix. If the cervix is not infected initially, the virus must spread locally, by autoinoculation, to the cervix for the individual to be at risk of developing cervical lesions. These can be single or multiple, and only develop in cells that have been infected. The production of progeny virions is usually limited to asymptomatic or low-grade lesions, as the full viral replication cycle is tied to the differentiation process.

In high-grade dysplasias, a more restricted number of ORF is expressed, primarily E6 and E7, and their expression is now found in the basal, proliferating layer of the epithelium. It has been difficult to quantify E6 protein, which is expressed at substantially lower levels. Both genes are expressed from a single promoter, with alternate splicing determining their relative level of expression, and progression to high-grade disease may be associated with a splicing pattern that favors E7 production. E6 and E7 regulate the expression of many genes.

During long-term infection these two viral genes appear to be the main drivers, via multiple mechanisms, for progression to high-grade dysplasia and cancer, by orchestrating a series of pathogenic changes. There is evidence that the quantity of E6, E7 mRNA is an early marker for dysplasia, with increasing levels correlating well with biopsy-proven CIN I, II, III and carcinoma in situ (Dürst et al., 1992).

Integration of HPV DNA, via nonhomologous recombination, represents a key change that appears to stabilize the high expression of E6/E7 and is associated with more severe lesions. Viral DNA integration is characteristically associated with deletion of large segments of the viral genome, and with transcription of sequences downstream from the integrated LCR.

In this integrated form, the E6 and E7 ORFs remain intact in the integrated viral DNA, and they can be transcribed from the LCR, which lies upstream in the integration site. Disruption of the viral E1 and E2 genes, as well as of downstream viral sequences, may permit higher levels of E6 and E7 transcription, whose RNA is stabilized following fusion to downstream cellular sequences. Cellular promoter elements near the integration site may also contribute to the increased viral gene expression. Whereas E6 and E7 are polyfunctional proteins and many of their biochemical activities have been found to contribute to the biological properties, the ability of high-risk E6 to inactivate p53 and of E7 to inactivate retinoblastoma protein pRb appears to be an essential property.

### Diagnosis and Treatment of HPV

Diagnosis. The approach used for the diagnosis of HPV infection may depend to a considerable degree on the underlying goal for making the diagnosis. These goals can include a determination of whether HPV is present, whether an active infection is present, which HPV type(s) is associated with the infection, and the degree of cellular atypia associated with the infection. If routine in vitro propagation of HPV from clinical samples were available, its isolation from productive infections, theoretically, would be possible. No such assays exist, however, and their utility would be limited by the fact that high-grade dysplasias and cancers do not produce infectious virus.

Serologic assays in an ELISA format that monitor the antibody response to L1 in 25 VLPs may have utility, and a high-throughput neutralization assay that appears to have comparable sensitivity has also been developed. These assays, which measure both current and past infection, are not sufficiently sensitive or specific, however, to be used for routine clinical diagnosis.

Nevertheless, sensitive, reproducible, and robust molecular assays have been developed to detect HPV DNA and RNA in cervical swabs and biopsy samples. The approaches for such assays include PCR consensus primers (or alternative amplification systems) that can be used in conjunction with a reverse line blot for specific hybridization, synthetic RNA probes that capture viral DNA, real time PCR, and microarrays. Most assays detect L1 DNA sequences, whereas others detect E6 or E7 DNA or RNA. There has been more experience with viral DNA detection, but measurement of viral RNA also appears be sensitive and specific. Several of the assays have been, or are in the process of being, rigorously validated, as is necessary for a clinical diagnostic test. Thus far, the hybrid capture II test, which detects a cocktail of high-risk HPV types, is the only test licensed thus far by the U.S. Food and Drug Administration (FDA). It is likely that this test will be superseded in the future by tests, such as hybrid capture III and others that identify specific HPV types.

Since its introduction in the 1950s, Pap smear screening has led to a substantial reduction in the incidence of cervical cancer. Even with technical improvements in Pap tests, we have entered a transition phase in which it is likely that HPV-based assays will supplant Pap smear screening as a primary screening assay, at least in some settings. Biomarkers other than HPV may also have the potential to contribute to identifying serious HPV infections, the most advanced being p16, which is elevated in response to the inactivation of pRb by E7 from high-risk viruses.

Treatment. In cervical HPV infection, treatment of low-grade dysplasia is not usually warranted, given that most of these lesions will clear spontaneously. High20 grade dysplasias represent precancerous lesions that are unlikely to resolve spontaneously, and their treatment is recommended to prevent cervical cancer. Depending on the setting, treatment of cervical dysplasia can be surgical, with cryotherapy, via loop electrosurgical excision repair, or by laser. In many instances, this approach prevents cervical cancer. HPV testing can be used in this setting, because most successfully treated cases become negative for HPV DNA, whereas incompletely treated cases may remain positive. Cervical cancer is treated by surgery, radiotherapy or chemotherapy, with early stage tumors having a better prognosis than more advanced tumors.

Therapeutic vaccine studies with preclinical models have shown some therapeutic efficacy with several nonstructural viral proteins, including E1, E2, E6, and E7, either as peptides, full-length proteins, or scrambled proteins. E6 and E7 have the theoretic advantage of being expressed in all stages of infection, including highgrade dysplasia and cancer, and most therapeutic vaccine studies have focused on these two proteins. It may be more difficult, however, to induce an effective therapeutic response in high-grade dysplasia and cancer, because immune parameters are more likely to be disregulated than in low-grade disease. Given what is now known about key molecular events in HPV infection, considerable potential exists for developing antiviral therapies against HPV. An antiviral that targeted a molecular activity common to all HPV types, or at least to a large number of them might have the theoretic advantage of being active against multiple types, in contrast to the predominantly type-specificity of most viral antigens. Antisense and ribozyme approaches may also have some potential, but their activity is likely to be type specific.

Two immunomodulatory agents, interferon and imiquimod, are approved for use against genital warts, although destructive therapy is often used to treat these lesions. In placebo-controlled trials, intralesional and parenteral interferon therapy was active against refractory genital warts, whereas topical imiquimod was also effective. Neither agent, however, cures more than two-thirds of treated patients.

As with other HPV infections, no specific antiviral therapy is available for nongenital warts. Most treatments are aimed at destroying the lesional tissue while causing as little long-term damage to the surrounding normal tissue. No treatment is likely to cure all warts, which has led to the wide range of therapies. At least partial regression can be obtained with many therapies, but even complete clearance that is then followed by recurrence is usually of limited clinical value. Traditional therapies include topical application of caustic agents (e.g., salicylic acid, podophyllin), cryotherapy, inhibitors of DNA synthesis (5-fluorouracil), and surgical therapy or laser treatment.

### Leukemia Inhibitory Factor (LIF) and its Utility in Treating HPV-Transformed Cells by Inhibiting LCR-Driven Transcription

LIF (leukemia inhibitory factor) is a 180-amino acid single-chain protein belonging to the IL-6 (interleukin-6) family of cytokines. IL-6 is known to repress transcription from the LCR (long control region) of HPV (human papillomavirus) (Kyo et al., 1993). Like IL-6, LIF negatively regulates transcription from the LCR (long control region) of HPV (human papillomavirus). However, while IL-6 acts as a growth factor in cervical and other malignancies (Iglesias et al., 1995), LIF represses both LCR-driven transcription and cell growth; in particular, LIF is able to reduce transcription of the HPV (human papillomavirus) oncogenes E6 and E7 through repression of the viral LCR (long control region), causing a substantial decrease in the abundance of the E6/E7 mRNA and, accordingly a reduced proliferation of HPV-transformed cells, as shown herein in the human cervical cancer cell lines CaSki E6 and SiHa E6. Since the HPV oncogenes are not found in uninfected human cells and are not required for any normal cellular process, they are an ideal target for therapy (Goodman & Wilbur, 2003).

Additionally, LIF has growth-inhibitory effects on human keratinocytes and cervical cancer cell lines. This is the first report of LIF-mediated inhibition of growth and HPV transcription in cervical cancer cell lines and keratinocyte cells. LIF has previously been tested for clinical applications in fertility treatment and the prevention of chemotherapy associated neuropathy. These studies have shown it to be well tolerated and safe for human use. LIF's ability to repress HPV transcription coupled with its inhibition of keratinocyte growth make us envision it for the treatment of HPV-associated cervical dysplasia, a condition for which practically no FDA approved nonsurgical intervention yet exists.

The human LIF gene is composed of three 30 exons and two introns. Exon 1 encodes the first 6 amino acid residues of the hydrophobic leader, with the remainder encoded by exon II. Exon II also encodes the first 44 residues of the mature protein. The C-terminal 136 amino acids are encoded by exon III. In short, the LIF precursor is 202 amino acids, the signal peptide is 22 amino acids, and the mature protein is 180 amino acids. Information about the nucleotide and amino acid sequence is available at ncbi.nlm.nih.gov/nuccore/208879451 and at ncbi.nlm.nih.gov/gene/3976.

The amino acid sequence of mature human LIF is

(SEQ ID NO: 1)
SPLPITPVNATCAIRHPCHNNLMNQIRSQLAQLNGSANALFILYYTAQG
EPFPNNLDKLCGPNVTDFPPFHANGTEKAKLVELYRIVVYLGTSLGNIT
RDQKILNPSALSLHSKLNATADILRGLLSNVLCRLCSKYHVGHVDVTYG
PDTSGKDVFQKKKLGCQLLGKYKQIIAVLAQAF.

Emfilermin is a recombinant human LIF (rhLIF) produced in Escherichia coli.

### Manipulating Proteins, DNA, and RNA

According to the central dogma of molecular biology, DNA is transcribed into RNA, and RNA is translated into protein; one gene makes one protein. DNA, or deoxyribonucleic acid, is a polynucleotide formed from covalently linked deoxyribonucleotide units. RNA, or ribonucleic acid, is a polynucleotide formed from covalently linked ribonucleotide units. Protein is a linear polymer of amino acids linked together by peptide bonds.

Isolating Cells and Growing Them in Culture. Although the organelles and large molecules in a cell can be visualized with microscopes, understanding how these components function requires a detailed biochemical analysis. Most biochemical procedures require that large numbers of cells be physically disrupted to gain access to their components. If the sample is a piece of tissue, composed of different types of cells, heterogeneous cell populations will be mixed together. To obtain as much information as possible about the cells in a tissue, biologists have developed ways of dissociating cells from tissues and separating them according to type. These manipulations result in a relatively homogeneous population of cells that can then be analyzed—either directly or after their number has been greatly increased by allowing the cells to proliferate in culture.

Cells Can Be Isolated from Intact Tissues. Intact tissues provide the most realistic source of material, as they represent the actual cells found within the body. The first step in isolating individual cells is to disrupt the extracellular matrix and cell-cell junctions that hold the cells together. For this purpose, a tissue sample is typically treated with proteolytic enzymes (such as trypsin and collagenase) to digest proteins in the extracellular matrix and with agents (such as ethylenediaminetetraacetic acid, or EDTA) that bind, or chelate, the Ca2+ on which cell-cell adhesion depends. The tissue can then be teased apart into single cells by gentle agitation.

For some biochemical preparations, the protein of interest can be obtained in sufficient quantity without having to separate the tissue or organ into cell types. In other cases, obtaining the desired protein requires enrichment for a specific cell type of interest. Several approaches are used to separate the different cell types from a mixed cell suspension. The most general cell-separation technique uses an antibody coupled to a fluorescent dye to label specific cells. An antibody is chosen that specifically binds to the surface of only one cell type in the tissue. The labeled cells can then be separated from the unlabeled ones in an electronic fluorescence-activated cell sorter. In this machine, individual cells traveling single file in a fine stream pass through a laser beam, and the fluorescence of each cell is rapidly measured. A vibrating nozzle generates tiny droplets, most containing either one cell or no cells.

The droplets containing a single cell are automatically given a positive or a negative charge at the moment of formation, depending on whether the cell they contain is fluorescent; they are then deflected by a strong electric field into an appropriate container. Occasional clumps of cells, detected by their increased light scattering, are left uncharged and are discarded into a waste container. Such machines can accurately select 1 fluorescent cell from a pool of 1000 unlabeled cells and sort several thousand cells each second.

Selected cells can also be obtained by carefully dissecting them from thin tissue slices that have been prepared for microscopic examination. In one approach, a tissue section is coated with a thin plastic film and a region containing the cells of interest is irradiated with a focused pulse from an infrared laser. This light pulse melts a small circle of the film, binding the cells underneath. These captured cells are then removed for further analysis. The technique, called laser capture microdissection, can be used to separate and analyze cells from different areas of a tumor, allowing their properties or molecular composition to be compared with neighboring normal cells. A related method uses a laser beam to directly cut out a group of cells and catapult them into an appropriate container for future analysis.

A uniform population of cells obtained by any of these or other separation methods can be used directly for biochemical analysis. After breaking open the cells by mechanical disruption, detergents, and other methods, cytoplasm or individual organelles can be extracted and then specific molecules purified.

Cells Can Be Grown in Culture. Although molecules can be extracted from whole tissues, this is often not the most convenient source of material, requiring, for example, early-morning trips to a slaughterhouse. The problem is not only a question of convenience. The livestock commonly used as organ sources are not amenable to genetic manipulation. Moreover, the complexity of intact tissues and organs is an inherent disadvantage when trying to purify particular molecules. Cells grown in culture provide a more homogeneous population of cells from which to extract material, and they are also much more convenient to work with in the laboratory. Given appropriate surroundings, most plant and animal cells can live, multiply, and even express differentiated properties in a tissue-culture dish. The cells can be watched continuously under the microscope or analyzed biochemically, and the effects of adding or removing specific molecules, such as hormones or growth factors, can be systematically explored. In addition, by mixing two cell types, the interactions between one cell type and another can be studied.

Experiments performed on cultured cells are sometimes carried out in vitro to contrast them with in vivo experiments using intact organisms. Cultures are most commonly made from suspensions of cells dissociated from tissues using the methods described earlier. Unlike bacteria, most tissue cells are not adapted to living suspended in fluid and require a solid surface on which to grow and divide. For cell cultures this support is usually provided by the surface of a plastic tissue-culture dish. Cells vary in their requirements, however, and many do not proliferate or differentiate unless the culture dish is coated with materials that cells like to adhere to, such as polylysine or extracellular matrix components.

Cultures prepared directly from the tissues of an organism are called primary cultures. These can be made with or without an initial fractionation step to separate different cell types. In most cases, cells in primary cultures can be removed from the culture dish and recultured repeatedly in so-called secondary cultures; in this way, they can be repeatedly subcultured (passaged) for weeks or months. Such cells often display many of the differentiated properties appropriate to their origin: fibroblasts continue to secrete collagen; cells derived from embryonic skeletal muscle fuse to form muscle fibers that contract spontaneously in the culture dish; nerve cells extend axons that are electrically excitable and make synapses with other nerve cells; and epithelial cells form extensive sheets with many of the properties of an intact epithelium.

Because these properties are maintained in culture, they are accessible to study in ways that are often not possible in intact tissues. Cell culture is not limited to animal cells. When a piece of plant tissue is cultured in a sterile medium containing nutrients and appropriate growth regulators, many of the cells are stimulated to proliferate indefinitely in a disorganized manner, producing a mass of relatively undifferentiated cells called a callus. If the nutrients and growth regulators are carefully manipulated, one can induce the formation of a shoot and then root apical meristems within the callus, and in many species, regenerate a whole new plant. Similar to animal cells, callus cultures can be mechanically dissociated into single cells, which will grow and divide as a suspension culture.

Eucaryotic Cell Lines Are a Widely Used Source of Homogeneous Cells. The cell cultures obtained by disrupting tissues tend to suffer from a problem—eventually the cells die. Most vertebrate cells stop dividing after a finite number of cell divisions in culture, a process called replicative cell senescence. Normal human fibroblasts, for example, typically divide only 25-40 times in culture before they stop. In these cells, the limited proliferation capacity reflects a progressive shortening and uncapping of the cell's telomeres, the repetitive DNA sequences and associated proteins that cap the ends of each chromosome. Human somatic cells in the body have turned off production of the enzyme, called telomerase, which normally maintains the telomeres, which is why their telomeres shorten with each cell division. Human fibroblasts can often be coaxed to proliferate indefinitely by providing them with the gene that encodes the catalytic subunit of telomerase; in this case, they can be propagated as an “immortalized” cell line. Some human cells, however, cannot be immortalized by this trick. Although their telomeres remain long, they still stop dividing after a limited number of divisions because the culture conditions eventually activate cell-cycle check-point mechanisms that arrest the cell cycle—a process sometimes called “culture shock.” In order to immortalize these cells, one has to do more than introduce telomerase. One must also inactivate the checkpoint mechanisms. This can be done by introducing certain cancer-promoting oncogenes, such as those derived from tumor viruses. Unlike human cells, most rodent cells do not turn off production of telomerase and therefore their telomeres do not shorten with each cell division. Therefore, if culture shock can be avoided, some rodent cell types will divide indefinitely in culture. In addition, rodent cells often undergo genetic changes in culture that inactivate their checkpoint mechanisms, thereby spontaneously producing immortalized cell lines.

Cell lines can often be most easily generated from cancer cells, but these cultures differ from those prepared from normal cells in several ways, and are referred to as transformed cell lines. Transformed cell lines often grow without attaching to a surface, for example, and they can proliferate to a much higher density in a culture dish. Similar properties can be induced experimentally in normal cells by transforming them with a tumor-inducing virus or chemical. The resulting transformed cell lines can usually cause tumors if injected into a susceptible animal (although it is usually only a small subpopulation, called cancer stem cells, that can do so). Both transformed and nontransformed cell lines are extremely useful in cell research as sources of very large numbers of cells of a uniform type, especially since they can be stored in liquid nitrogen at −196° C. for an indefinite period and retain their viability when thawed. It is important to keep in mind, however, that the cells in both types of cell lines nearly always differ in important ways from their normal progenitors in the tissues from which they were derived.

Some widely used cell lines are as follows, listing cell line and cell type (and origin): 3T3, fibroblast (mouse); BHK, fibroblast (Syrian hamster); MDCK, epithelial cell (dog); HeLa, epithelial cell (human); PtK1, epithelial cell (rat kangaroo); L6, myoblast (rat); PC12, chromaffin cell (rat); SP2, plasma cell (mouse); COS, kidney (monkey); 293 kidney (human, transformed with adenovirus); CHO, ovary (Chinese hamster); DT40, lymphoma cell for efficient targeted recombination (chick); R1, embryonic stem cell (mouse); E14.1, embryonic stem cell (mouse); H1, H9, embryonic stem cell (human); S2, macrophage-like cell (Drosophila); BY2, undifferentiated meristematic cell (tobacco).

Embryonic Stem Cells Are Promising Cell Lines. Embryonic stem (ES) cells are cells that are derived from an embryo. These cultured cells can give rise to all of the cell types of the body. ES cells are harvested from the inner cell mass of an early embryo and can be maintained indefinitely as stem cells in culture. If they are put back into an embryo, they will integrate perfectly and differentiate to suit whatever environment they find themselves. The cells can also be kept in culture as an immortal cell line; they can then be supplied with different hormones or growth factors to encourage them to differentiate into specific cell types.

Hybridoma Cell Lines Are Factories That Produce Monoclonal Antibodies. An antibody, also called an immunoglobulin (Ig), is a protein produced by cells of the immune system in response to an antigen. Antibodies are particularly useful tools for cell biology. Their great specificity allows precise visualization of selected proteins among the many thousands that each cell typically produces. Antibodies are often produced by inoculating animals with the protein of interest and subsequently isolating the antibodies specific to that protein from the serum of the animal. However, only limited quantities of antibodies can be obtained from a single inoculated animal, and the antibodies produced will be a heterogeneous mixture of antibodies that recognize a variety of different determinants on a macromolecule that differs from animal to animal. Moreover, antibodies specific for the antigen will constitute only a fraction of the antibodies found in the serum. An alternative technology, which allows the production of an infinite quantity of identical antibodies and greatly increases the specificity and convenience of antibody-based methods, is the production of monoclonal antibodies by hybridoma cell lines. This technology has facilitated the production of antibodies for use as tools in cell biology, as well as for the diagnosis and treatment of certain diseases. The procedure requires hybrid cell technology, and it involves propagating a clone of cells from a single antibody-secreting B lymphocyte to obtain a homogeneous preparation of antibodies in large quantities. B lymphocytes normally have a limited life-span in culture, but individual antibody-producing B lymphocytes from an immunized mouse or rat, when fused with cells derived from a transformed B lymphocyte cell line, can give rise to hybrids that have both the ability to make a particular antibody and the ability to multiply indefinitely in culture. These hybridomas are propagated as individual clones, each of which provides a permanent and stable source of a single type of monoclonal antibody.

Each type of monoclonal antibody recognizes a single determinant of an antigen—for example, a particular cluster of five or six amino acid side chains on the surface of a protein. Their uniform specificity makes monoclonal antibodies much more useful than conventional antisera for most purposes. Hybridomas are prepared that secrete monoclonal antibodies against a particular antigen by immunizing a mouse with antigen X and fusing the cells that make antibodies (including the cell making anti-X antibody) obtained from the spleen with a mutant cell line derived from a tumor of B lymphocytes. The selective growth medium used after the cell fusion step contains an inhibitor (aminopterin) that blocks the normal biosynthetic pathways by which nucleotides are made. The cells must therefore use a bypass pathway to synthesize their nucleic acids. This pathway is defective in the mutant cell line derived from the B cell tumor, but it is intact in the normal cells obtained from the immunized mouse. Nevertheless, the normal B lymphocytes will die after a few days in culture. Because neither cell type used for the initial fusion can survive and proliferate on its own, only the hybridoma cells do so. The hybridoma cells are cloned by limiting dilution, the supernatants tested for anti-X antibodies, and positive clones selected that provide a continuing source of anti-X antibody.

An important advantage of the hybridoma technique is that monoclonal antibodies can be made against molecules that constitute only a minor component of a complex mixture. In an ordinary antiserum made against such a mixture, the proportion of antibody molecules that recognize the minor component would be too small to be useful. But if the B lymphocytes that produce the various components of this antiserum are made into hybridomas, it becomes possible to screen individual hybridoma clones from the large mixture to select one that produces the desired type of monoclonal antibody and to propagate the selected hybridoma indefinitely so as to produce that antibody in unlimited quantities. In principle, therefore, a monoclonal antibody can be made against any protein in a biological sample. Once an antibody has been made, it can be used to localize the protein in cells and tissues, to follow its movement, and to purify the protein of interest.

Purifying Proteins. The challenge of isolating a single type of protein from the thousands of other proteins present in a cell is a formidable one, but must be overcome in order to produce purified proteins. Recombinant DNA technology can enormously simplify this task by “tricking” cells into producing large quantities of a given protein, thereby making its purification a little easier. Whether the source of the protein is an engineered cell or a natural tissue, a purification procedure usually starts with subcellular fractionation to reduce the complexity of the material, and is then followed by purification steps of increasing specificity.

Cells Can Be Separated into Their Component Fraction. In order to purify a protein, it must first be extracted from inside the cell. Cells can be broken up in various ways: they can be subjected to osmotic shock or ultrasonic vibration, forced through a small orifice, or ground up in a blender. These procedures break many of the membranes of the cell (including the plasma membrane and endoplasmic reticulum) into fragments that immediately reseal to form small closed vesicles. If carefully carried out, however, the disruption procedures leave organelles such as nuclei, mitochondria, the Golgi apparatus, lysosomes, and peroxisomes largely intact. The suspension of cells is thereby reduced to a thick slurry (called a homogenate or extract) that contains a variety of membrane-enclosed organelles, each with a distinctive size, charge and density. Provided that the homogenization medium has been carefully chosen (by trial and error for each organelle), the various components—including the vesicles derived from the endoplasmic reticulum, called microsomes—retain most of their original biochemical properties.

The different components of the homogenate must then be separated. Such cell fractionations became possible only after the commercial development of an instrument known as the preparative ultracentrifuge, which rotates extracts of broken cells at high speeds. This treatment separates cell components by size and density: in general, the largest units experience the largest centrifugal force and move the most rapidly. At relatively low speed, large components such as nuclei sediment to form a pellet at the bottom of the centrifuge tube; at slightly higher speed, a pellet of mitochondria is deposited; and at even higher speeds and with longer periods of centrifugation, first the small closed vesicles and then the ribosomes can be collected. All of these fractions are impure, but many of the contaminants can be removed by resuspending the pellet and repeating the centrifugation procedure several times.

Centrifugation is the first step in most fractionations, but it separates only components that differ greatly in size. A finer degree of separation can be achieved by layering the homogenate in a thin band on top of a dilute salt solution that fills a centrifuge tube. When centrifuged, the various components in the mixture move as a series of distinct bands through the salt solution, each at a different rate, in a process called velocity sedimentation. For the procedure to work effectively, the bands must be protected from convective mixing, which would normally occur whenever a denser solution (for example, one containing organelles) finds itself on top of a lighter one (the salt solution). This is achieved by augmenting the solution in the tube with a shallow gradient of sucrose prepared by a special mixing device. The resulting density gradient—with the dense end at the bottom of the tube—keeps each region of the salt solution denser than any solution above it, and it thereby prevents convective mixing from distorting the separation.

When sedimented through such dilute sucrose gradients, different cell components separate into distinct bands that can be collected individually. The relative rate at which each component sediments depends primarily on its size and shape—normally being described in terms of its sedimentation coefficient, or S value. Present-day ultracentrifuges rotate at speeds of up to 80,000 rpm and produce forces as high as 500,000 times gravity. These enormous forces drive even small macromolecules, such as tRNA molecules and simple enzymes, to sediment at an appreciable rate and allow them to be separated from one another by size. The ultracentrifuge is also used to separate cell components on the basis of their buoyant density, independently of their size and shape. In this case the sample is sedimented through a steep density gradient that contains a very high concentration of sucrose or cesium chloride. Each cell component begins to move down the gradient, but it eventually reaches a position where the density of the solution is equal to its own density. At this point the component floats and can move no farther. A series of distinct bands is thereby produced in the centrifuge tube, with the bands closest to the bottom of the tube containing the components of highest buoyant density. This method, called equilibrium sedimentation, is so sensitive that it can separate macromolecules that have incorporated heavy isotopes, such as 13C or 15N, from the same macromolecules that contain the lighter, common isotopes (12C or 14N).

Cell Extracts Provide Accessible Systems to Study Cell Functions. Cell extracts isolated in the ultracentrifuge have contributed to our understanding of cell functions. They have played a good role in the study of cell processes. Cell extracts also provide, in principle, the starting material for the separation of proteins.

Proteins Can Be Separated by Chromatography. Proteins are often fractionated by column chromatography, in which a mixture of proteins in solution is passed through a column containing a porous solid matrix. The different proteins are retarded to different extents by their interaction with the matrix, such as cellulose, and they can be collected separately as they flow out of the bottom of the column. Depending on the choice of matrix, proteins can be separated according to their charge (ion-exchange chromatography), their hydrophobicity (hydrophobic chromatography), their size (gel-filtration chromatography), or their ability to bind to particular small molecules or to other macromolecules (affinity chromatography).

Many types of matrices are commercially available. Ion exchange columns are packed with small beads that carry either a positive or a negative charge, so that proteins are fractionated according to the arrangement of charges on their surface. Hydrophobic columns are packed with beads from which hydrophobic side chains protrude, selectively retarding proteins with exposed hydrophobic regions. Gelfiltration columns, which separate proteins according to their size, are packed with tiny porous beads: molecules that are small enough to enter the pores linger inside successive beads as they pass down the column, while larger molecules remain in the solution flowing between the beads and therefore move more rapidly, emerging from the column first. Inhomogeneities in the matrices (such as cellulose), which cause an uneven flow of solvent through the column, limit the resolution of conventional column chromatography.

Special chromatography resins (usually silica-based) composed of tiny spheres (3-10 μm in diameter) can be packed with a special apparatus to form a uniform column bed. Such high-performance liquid chromatography (HPLC) columns attain a high degree of resolution. In HPLC, the solutes equilibrate very rapidly with the interior of the tiny spheres, and so solutes with different affinities for the matrix are efficiently separated from one another even at very fast flow rates. HPLC is therefore the method of choice for separating many proteins and small molecules.

Affinity Chromatography Exploits Specific Binding Sites on Proteins. If one starts with a complex mixture of proteins, the types of column chromatography just discussed do not produce very highly purified fractions: a single passage through the column generally increases the proportion of a given protein in the mixture no more than twentyfold. Because most individual proteins represent less than 1/1000 of the total cell protein, it is usually necessary to use several different types of columns in succession to attain sufficient purity. A more efficient procedure, known as affinity chromatography, takes advantage of the biologically important binding interactions that occur on protein surfaces. If a substrate molecule is covalently coupled to an inert matrix such as a polysaccharide bead, the enzyme that operates on that substrate will often be specifically retained by the matrix and can then be eluted (washed out) in nearly pure form. Likewise, short DNA oligonucleotides of a specifically designed sequence can be immobilized in this way and used to purify DNA-binding proteins that normally recognize this sequence of nucleotides in chromosomes. Alternatively, specific antibodies can be coupled to a matrix to purify protein molecules recognized by the antibodies. Because of the great specificity of all such affinity columns, 1000- to 10.000-fold purifications can sometimes be achieved in a single pass.

Three types of matrices commonly used for chromatography can be compared as follows. In ion-exchange chromatography, the insoluble matrix carries ionic charges that retard the movement of molecules of opposite charge. Matrices used for separating proteins include diethylaminoethylcellulose (DEAE-cellulose), which is positively charged, and carboxymethylcellulose (CM-cellulose) and phosphocellulose, which are negatively charged.

Analogous-matrices based on agarose or other polymers are also frequently used. The strength of the association between the dissolved molecules and the ion-exchange matrix depends on both the ionic strength and the pH of the solution that is passing down the column, which may therefore be varied systematically to achieve an effective separation. In gel-filtration chromatography, the matrix is inert but porous. Molecules that are small enough to penetrate into the matrix are thereby delayed and travel more slowly through the column than larger molecules that cannot penetrate. Beads of cross-linked polysaccharide (dextran, agarose or acrylamide) are available commercially in a wide range of pore sizes, making them suitable for the fractionation of molecules of various molecular weights, from less than 500 daltons to more than 5×10̂6 daltons. Affinity chromatography uses an insoluble matrix that is covalently linked to a specific ligand, such as an antibody molecule or an enzyme substrate, that will bind a specific protein.

Enzyme molecules that bind to immobilized substrates on such columns can be eluted with a concentrated solution of the free form of the substrate molecule, while molecules that bind to immobilized antibodies can be eluted by dissociating the antibody-antigen complex with concentrated salt solutions or solutions of high or low pH. High degrees of purification can be achieved in a single pass through an affinity column.

Genetically-Engineered Tags Provide an Easy Way to Purify Proteins. Using recombinant DNA methods, a gene can be modified to produce its protein with a special recognition tag attached to it, so as to make subsequent purification of the protein by affinity chromatography simple and rapid. Often the recognition tag is itself an antigenic determinant, or epitope, which can be recognized by a highly specific antibody. The antibody can then be used both to localize the protein in cells and to purify it. Other types of tags are specifically designed for protein purification. For example, the amino acid histidine binds to certain metal ions, including nickel and copper. If genetic engineering techniques are used to attach a short string of histidines to one end of a protein, the slightly modified protein can be retained selectively on an affinity column containing immobilized nickel ions. Metal affinity chromatography can thereby be used to purify the modified protein from a complex molecular mixture.

In other cases, an entire protein is used as the recognition tag. When cells are engineered to synthesize the small enzyme glutathione S-transferase (GST) attached to a protein of interest, the resulting fusion protein can be purified from the other contents of the cell with an affinity column containing glutathione, a substrate molecule that binds specifically and tightly to GST. If the purification is carried out under conditions that do not disrupt protein-protein interactions, the fusion protein can be isolated in association with the proteins it interacts with inside the cell. As a further refinement of purification methods using recognition tags, an amino acid sequence that forms a cleavage site for a highly specific proteolytic enzyme can be engineered between the protein of choice and the recognition tag. Because the amino acid sequences at the cleavage site are very rarely found by chance in proteins, the tag can later be cleaved off without destroying the purified protein. This type of specific cleavage is used in an especially powerful purification methodology known as tandem affinity purification tagging (tap-tagging). Here, one end of a protein is engineered to contain two recognition tags that are separated by a protease cleavage site. The tag on the very end of the construct is chosen to bind irreversibly to an affinity column, allowing the column to be washed extensively to remove all contaminating proteins. Protease cleavage then releases the protein, which is then further purified using the second tag. Because this two-step strategy provides an especially high degree of protein purification with relatively little effort, it is used extensively in cell biology.

Purified Cell-free Systems Are Instrumental for the Precise Dissection of Molecular Functions. It is good to study biological processes free from all of the complex side reactions that occur in a living cell by using purified cell-free systems. To make this possible, cell homogenates are fractionated with the aim of purifying each of the individual macromolecules that are needed to catalyze a biological process of interest. For example, the experiments to decipher the mechanisms of protein synthesis began with a cell homogenate that could translate RNA molecules to produce proteins. Fractionation of this homogenate, step by step, produced in turn the ribosomes, tRNAs, and various enzymes that together constitute the protein-synthetic machinery. Once individual pure components were available, each could be added or withheld separately to define its exact role in the overall process. A major goal for cell biologists is the reconstitution of biological process in a purified cell-free system. In this way can one define all of the components needed for the process and control their concentrations, as required to work out their precise mechanism of action. Although much remains to be done, a great deal of what we know today about the molecular biology of the cell has been discovered by studies in such cell-free systems. They have been used, for example, to decipher the molecular details of DNA replication and DNA transcription, RNA splicing, protein translation, and many other processes that occur in cells.

Analyzing Proteins. Proteins perform most processes in cells: they catalyze metabolic reactions, use nucleotide hydrolysis to do mechanical work, and serve as the major structural elements of the cell. The great variety of protein structures and functions has stimulated the development of a multitude of techniques to study them.

Proteins Can Be Separated by SDS Polyacrylamide-Gel Electrophoresis. Proteins usually possess a net positive or negative charge, depending on the mixture of charged amino acids they contain. An electric field applied to a solution containing a protein molecule causes the protein to migrate at a rate that depends on its net charge and on its size and shape. The most popular application of this property is SDS polyacrylamide-gel electrophoresis (SDS-PAGE). It uses a highly cross-linked gel of polyacrylamide as the inert matrix through which the proteins migrate. The gel is prepared by polymerization of monomers; the pore size of the gel can be adjusted so that it is small enough to retard the migration of the protein molecules of interest.

The proteins themselves are not in a simple aqueous solution but in one that includes a powerful negatively charged detergent, sodium dodecyl sulfate, or SDS. Because this detergent binds to hydrophobic regions of the protein molecules, causing them to unfold into extended polypeptide chains, the individual protein molecules are released from their associations with other proteins or lipid molecules and rendered freely soluble in the detergent solution. In addition, a reducing agent such as β-mercaptoethanol is usually added to break any S-S linkages in the proteins, so that all of the constituent polypeptides in multisubunit proteins can be analyzed separately.

What happens when a mixture of SDS-solubilized proteins is run through a slab of polyacrylamide gel? Each protein molecule binds large numbers of the negatively charged detergent molecules, which mask the protein's intrinsic charge and cause it to migrate toward the positive electrode when a voltage is applied. Proteins of the same size tend to move through the gel with similar speeds because (1) their native structure is completely unfolded by the SDS, so that their shapes are the same, and (2) they bind the same amount of SDS and therefore have the same amount of negative charge. Larger proteins, with more charge, are subjected to larger electrical forces and also to a larger drag. In free solution, the two effects would cancel out, but, in the mesh of the polyacrylamide gel, which acts as a molecular sieve, large proteins are retarded much more than small ones. As a result, a complex mixture of proteins is fractionated into a series of discrete protein bands arranged in order of molecular weight. The major proteins are readily detected by staining the proteins in the gel with a dye such as Coomassie blue. Even minor proteins are seen in gels treated with a silver or gold stain, so that as little as 10 ng of protein can be detected in a band. SDS-PAGE is widely used because it can separate all types of proteins, including those that are normally insoluble in water—such as the many proteins in membranes. And because the method separates polypeptides by size, it provides information about the molecular weight and the subunit composition of proteins. A photograph of a Coomasie-stained gel is handy for memorializing an analysis of each of the successive stages in the purification of a protein.

Specific Proteins Can Be Detected by Blotting with Antibodies. A specific protein can be identified after its fractionation on a polyacrylamide gel by exposing all the proteins present on the gel to a specific antibody that has been coupled to a radioactive isotope, to an easily detectable enzyme, or to a fluorescent dye. For convenience, this procedure is normally carried out after transferring (by “blotting”) all of the separated proteins present in the gel onto a sheet of nitrocellulose paper or nylon membrane. Placing the membrane over the gel and driving the proteins out of the gel with a strong electric field transfers the protein onto the membrane. The membrane is then soaked in a solution of labeled antibody to reveal the protein of interest. This method of detecting proteins is called Western blotting, or immunoblotting.

Mass Spectrometry Provides a Highly Sensitive Method for Identifying Unknown Proteins. A frequent problem in cell biology and biochemistry is the identification of a protein or collection of proteins that has been obtained by one of the purification procedures for proteins. Because the genome sequences of most common experimental organisms are now known, catalogues of all the proteins produced in those organisms are available. The task of identifying an unknown protein (or collection of unknown proteins) thus reduces to matching some of the amino acid sequences present in the unknown sample with known catalogued genes. This task is now performed almost exclusively by using mass spectrometry in conjunction with computer searches of databases. Charged particles have very precise dynamics when subjected to electrical and magnetic fields in a vacuum. Mass spectrometry exploits this principle to separate ions according to their mass-to-charge ratio. It is an enormously sensitive technique. It requires very little material and is capable of determining the precise mass of intact proteins and of peptides derived from them by enzymatic or chemical cleavage. Masses can be obtained with great accuracy, often with an error of less than one part in a million. The most commonly used form of the technique is called matrix-assisted laser desorption ionization-time-of-flight spectrometry (MALDI-TOF). In this approach, the proteins in the sample are first broken into short peptides. These peptides are mixed with an organic acid and then dried onto a metal or ceramic slide.

A laser then blasts the sample, ejecting the peptides from the slide in the form of an ionized gas, in which each molecule carries one or more positive charges. The ionized peptides are accelerated in an electric field and fly toward a detector. Their mass and charge determines the time it takes them to reach the detector: large peptides move more slowly, and more highly charged molecules move more quickly. By analyzing those ionized peptides that bear a single charge, the precise masses of peptides present in the original sample can be determined. MALDI-TOF can also be used to accurately measure the mass of intact proteins as large as 200,000 daltons. This information is then used to search genomic databases, in which the masses of all proteins and of all their predicted peptide fragments have been tabulated from the genomic sequences of the organism. An unambiguous match to a particular open reading frame can sometimes be made by knowing the mass of only a few peptides derived from a given protein.

MALDI-TOF provides accurate molecular weight measurements for proteins and peptides. Moreover, by employing two mass spectrometers in tandem (an arrangement known as MS/MS), it is possible to directly determine the amino acid sequences of individual peptides in a complex mixture. As described above, the protein sample is first broken down into smaller peptides, which are separated from each other by mass spectrometry. Each peptide is then further fragmented through collisions with high-energy gas atoms. This method of fragmentation preferentially cleaves the peptide bonds, generating a ladder of fragments, each differing by a single amino acid. The second mass spectrometer then separates these fragments and displays their masses. The amino acid sequence of a peptide can then be deduced from these differences in mass.

MS/MS is particularly useful for detecting and precisely mapping post translational modifications of proteins, such as phosphorylations or acetylations. Because these modifications impart a characteristic mass increase to an amino acid, they are easily detected by mass spectrometry. In combination with rapid purification techniques, mass spectrometry has emerged as a powerful method for detecting posttranslational modifications of proteins and the identity of proteins present in mixtures of proteins.

Two-Dimensional Separation Methods are Especially Powerful. Because different proteins can have similar sizes, shapes, masses, and overall charges, most separation techniques such as SDS polyacrylamide-gel electrophoresis or ion-exchange chromatography cannot typically display all the proteins in a cell or even in an organelle. In contrast, two-dimensional gel electrophoresis, which combines two different separation procedures, can resolve up to 2000 proteins—the total number of different proteins in a simple bacterium—in the form of a two-dimensional protein map. In the first step, the proteins are separated by their intrinsic charges. The sample is dissolved in a small volume of a solution containing a non-ionic (uncharged) detergent, together with β-mercaptoethanol and the denaturing reagent urea. This solution solubilizes, denatures, and dissociates all the polypeptide chains but leaves their intrinsic charge unchanged. The polypeptide chains are then separated in a pH gradient by a procedure called isoelectric focusing, which takes advantage of the variation in the net charge on a protein molecule with the pH of its surrounding solution. Every protein has a characteristic isoelectric point, the pH at which the protein has no net charge and therefore does not migrate in an electric field.

In isoelectric focusing, proteins are separated electrophoretically in a narrow tube of polyacrylamide gel in which a gradient of pH is established by a mixture of special buffers. Each protein moves to a position in the gradient that corresponds to its isoelectric point and remains there. This is the first dimension of two-dimensional polyacrylamide-gel electrophoresis. In the second step, the narrow gel containing the separated proteins is again subjected to electrophoresis but in a direction that is at a right angle to the direction used in the first step. This time SDS is added, and the proteins separate according to their size, as in one-dimensional SDS-PAGE: the original narrow gel is soaked in SDS and then placed on one edge of an SDS polyacrylamide-gel slab, through which each polypeptide chain migrates to form a discrete spot. This is the second dimension of two-dimensional polyacrylamide-gel electrophoresis. The only proteins left unresolved are those that have both identical sizes and identical isoelectric points, a relatively rare situation. Even trace amounts of each polypeptide chain can be detected on the gel by various staining procedures—or by autoradiography if the protein sample was initially labeled with a radioisotope. The technique has such great resolving power that it can distinguish between two proteins that differ in only a single charged amino acid.

A different, even more powerful, “two-dimensional” technique is now available when the aim is to determine all of the proteins present in an organelle or another complex mixture of proteins. Because the technique relies on mass spectroscopy, it requires that the proteins be from an organism with a completely sequenced genome. First, the mixture of proteins present is digested with trypsin to produce short peptides. Next, these peptides are separated by a series of automated liquid chromatography steps. As the second dimension, each separated peptide is fed directly into a tandem mass spectrometer (MS/MS) that allows its amino acid sequence, as well as any post-translational modifications, to be determined. This arrangement, in which a tandem mass spectrometer (MS/MS) is attached to the output of an automated liquid chromatography (LC) system, is referred to as LC-MS/MS. It is now becoming routine to subject an entire organelle preparation to LC-MS/MS analysis and to identify hundreds of proteins and their modifications. Of course, no organelle isolation procedure is perfect, and some of the proteins identified will be contaminating proteins. These can conceivably be excluded by analyzing neighboring fractions from the organelle purification and “subtracting” them out from the peak organelle fractions.

Hydrodynamic Measurements Reveal the Size and Shape of a Protein Complex. Most proteins in a cell act as part of larger complexes, and knowledge of the size and shape of these complexes often leads to insights regarding their function. This information can be obtained in several important ways. Sometimes, a complex can be directly visualized using electron microscopy. A complementary approach relies on the hydrodynamic properties of a complex, that is, its behavior as it moves through a liquid medium. Usually, two separate measurements are made. One measure is the velocity of a complex as it moves under the influence of a centrifugal field produced by an ultracentrifuge. The sedimentation constant (or S-value) obtained depends on both the size and the shape of the complex and does not, by itself, convey especially useful information. However, once a second hydrodynamic measurement is Performed—by charting the migration of a complex through a gel-filtration chromatography column—both the approximate shape of a complex and its molecular weight can be calculated.

Molecular weight can also be determined more directly by using an analytical ultracentrifuge, a complex device that allows protein absorbance measurements to be made on a sample while it is subjected to centrifugal forces. In this approach, the sample is centrifuged until it reaches equilibrium, where the centrifugal force on a protein complex exactly balances its tendency to diffuse away. Because this balancing point is dependent on a complex's molecular weight but not on its particular shape, the molecular weight can be directly calculated, as needed to determine the stoichiometry of each protein in a protein complex.

Sets of Interacting Proteins Can Be Identified by Biochemical Methods. Because most proteins in the cell function as part of complexes with other proteins, a preliminary way to begin to characterize the biological role of an unknown protein is to identify all of the other proteins to which it specifically binds. One method for identifying proteins that bind to one another tightly is coimmunoprecipitation. In this case, an antibody recognizes a specific target protein; reagents that bind to the antibody and are coupled to a solid matrix then drag the complex out of solution to the bottom of a test tube. If the original target protein is associated tightly enough with another protein when it is captured by the antibody, the partner precipitates as well. This method is useful for identifying proteins that are part of a complex inside cells, including those that interact only transiently—for example, when extracellular signal molecules stimulate cells. Another method frequently used to identify a protein's binding partners is protein affinity chromatography. To employ this technique to capture interacting proteins, a target protein is attached to polymer beads that are packed into a column. When the proteins in a cell extract are washed through this column, those proteins that interact with the target protein are retained by the affinity matrix. These proteins can then be eluted and their identity determined by mass spectrometry.

In addition to capturing protein complexes on columns or in test tubes, researchers are developing high-density protein arrays to investigate protein interactions. These arrays, which contain thousands of different proteins or antibodies spotted onto glass slides or immobilized in tiny wells, allow one to examine the biochemical activities and binding profiles of a large number of proteins at once. For example, if one incubates a fluorescently labeled protein with arrays containing thousands of immobilized proteins, the spots that remain fluorescent after extensive washing each contain a protein to which the labeled protein specifically binds.

Protein-Protein Interactions Can Also Be Identified by a Two-Hybrid Technique in Yeast. The yeast two-hybrid system is another way, besides a biochemical approach, to reveal protein-protein interactions. The technique takes advantage of the modular nature of gene activator proteins. These proteins both bind to specific DNA sequences and activate gene transcription, and these activities are often performed by two separate protein domains. Using recombinant DNA techniques, two such protein domains are used to create separate “bait” and “prey” fusion proteins. To create the “bait” fusion protein, the DNA sequence that codes for a target protein is fused with DNA that encodes the DNA-binding domain of a gene activator protein. When this construct is introduced into yeast, the cells produce the fusion protein, with the target protein attached to this DNA-binding domain. This fusion protein binds to the regulatory region of a reporter gene, where it serves as “bait” to fish for proteins that interact with the target protein.

To search for potential binding partners (potential prey for the bait), the candidate proteins also have to be constructed as fusion proteins: DNA encoding the activation domain of a gene activator protein is fused to a large number of different genes. Members of this collection of genes—encoding potential “prey”—are introduced individually into yeast cells containing the bait. If the yeast cell receives a DNA clone that expresses a prey partner for the bait protein, the two halves of a transcriptional activator are united, switching on the reporter gene. This ingenious technique sounds complex, but the two-hybrid system is straightforward to use in the laboratory. Although the protein-protein interactions occur in the yeast cell nucleus, proteins from every part of the cell and from candidate organisms can be studied in this way. The two-hybrid system has been scaled up to map the interactions that occur among many of the proteins an organism produces. In this case, a set of bait and prey fusions is produced for each cell protein, and every bait/prey combination can be monitored. In this way protein interaction maps have been generated for many proteins.

Combining Data Derived from Different Techniques Produces Reliable Protein-Interaction Maps. Extensive protein-interaction maps can be very useful for identifying the functions of proteins. For this reason, both the two-hybrid method and the biochemical technique known as tap-tagging have been automated to determine the interactions between thousands of proteins. Unfortunately, different results are found in different experiments, and many of the interactions detected in one laboratory are not detected in another. Therefore, the most useful protein-interaction maps are those that combine data from many experiments, requiring that each interaction in the map be confirmed by more than one technique.

Optical Methods Can Monitor Protein Interactions in Real Time. Once two proteins—or a protein and a small molecule—are known to associate, it becomes useful to characterize their interaction in more detail. Proteins can associate with each other more or less permanently (like the subunits of RNA polymerase), or engage in transient encounters that may last only a few milliseconds (like a protein kinase and its substrate). To understand how a protein functions inside a cell, we seek to determine how tightly it binds to other proteins, how rapidly it dissociates from them, and how covalent modifications, small molecules, or other proteins influence these interactions. Such studies of protein dynamics often employ optical methods.

Certain amino acids (for example, tryptophan) exhibit weak fluorescence that can be detected with sensitive fluorimeters. In many cases, the fluorescence intensity, or the emission spectrum of fluorescent ammo acids located in a protein-protein interface, will change when the proteins associate. When this change can be detected by fluorimetry, it provides a sensitive and quantitative measure of protein binding. A particularly useful method for monitoring the dynamics of a protein's binding to other molecules is called surface plasmon resonance (SPR). The SPR method has been used to characterize a wide variety of molecular interactions, including antibody-antigen binding, ligand-receptor coupling, and the binding of proteins to DNA, carbohydrates, small molecules, and other proteins. SPR detects binding interactions by monitoring the reflection of a beam of light off the interface between an aqueous solution of potential binding molecules and a biosensor surface carrying an immobilized bait protein. The bait protein is attached to a very thin layer of metal that coats one side of a glass prism. A light beam is passed through the prism; at a certain angle, called the resonance angle, some of the energy from the light interacts with the cloud of electrons in the metal film, generating a plasmon—an oscillation of the electrons at right angles to the plane of the film, bouncing up and down between its upper and lower surfaces like a weight on a spring.

The plasmon, in turn, generates an electrical field that extends a short distance—about the wavelength of the light—above and below the metal surface. Any change in the composition of the environment within the range of the electrical field will cause a measurable change in the resonance angle. To measure binding, a solution containing proteins (or other molecules) that might interact with the immobilized bait protein is allowed to flow past the biosensor surface. Proteins binding to the bait change the composition of the molecular complexes on the metal surface, causing a change in the resonance angle. The changes in the resonance angle are monitored in real time and reflect the kinetics of the association—or dissociation—of molecules with the bait protein. The association rate (k on) is measured as the molecules interact, and the dissociation rate (k off) is determined as buffer washes the bound molecules from the sensor surface. A binding constant (K) is calculated by dividing k off by k on. In addition to determining the kinetics, SPR can be used to determine the number of molecules that are bound in each complex: the magnitude of the SPR signal change is proportional to the mass of the immobilized complex.

The SPR method is particularly useful because it requires only small amounts of the protein, the protein does not have to be labeled in anyway, and the interactions of the protein with other molecules can be monitored in real time. A third optical method for probing protein interactions uses green fluorescent protein and its derivatives of different colors. In this application, two proteins of interest are each labeled with a different fluorochrome, such that the emission spectrum of one fluorochrome overlaps the absorption spectrum of the second fluorochrome. If the two proteins—and their attached fluorochromes—come very close to each other (within about 1-10 nm), the energy of the absorbed light is transferred from one fluorochrome to the other. The energy transfer, called fluorescence resonance energy transfer (FRET), is determined by illuminating the first fluorochrome and measuring emission from the second. This technique is especially powerful because, when combined with fluorescence microscopy, it can be used to characterize protein-protein interactions at specific locations inside living cells.

Some Techniques Can Monitor Single Molecules. The light microscope can resolve details about 0.2 μm apart. Living cells are seen clearly in a phase-contrast or a differential-interference-contrast microscope. Images can be enhanced and analyzed by digital techniques. Intact tissues are usually fixed and sectioned before microscopy. Specific molecules can be located in cells by fluorescence microscopy. Antibodies can be used to detect specific molecules. Imaging of complex three-dimensional objects is possible with the optical microscope. The confocal microscope produces optical sections by excluding out-of focus light. Fluorescent proteins can be used to tag individual proteins in living cells and organisms. Light-emitting indicators can measure rapidly changing intracellular ion concentrations. Several strategies are available by which membrane-impermeant substances can be introduced into cells. Light can be used not only to image microscopic objects but also to manipulate them as well. Molecules can be labeled with radioisotopes and the radioisotopes used to trace molecules in cells and organisms. The electron microscope resolves the fine structure of the cell. Biological specimens require special preparation for the electron microscope. Specific macromolecules can be localized by immunogold electron microscopy. Images of surfaces can be obtained by canning electron microscopy. Metal shadowing allows surface features to be examined at high resolution by transmission electron microscopy. Negative staining and cryoelectron microscopy both allow macromolecules to be viewed at high resolution. Multiple images can be combined to increase resolution. Different views of a single object can be combined to give a three-dimensional reconstruction.

Protein Function Can Be Selectively Disrupted With Small Molecules. Chemical inhibitors have contributed to the development of cell biology. Small organic molecules are carbon-based compounds that have molecular weights in the range 100-1000 and contain up to 30 or so carbon atoms. In the past, small molecules were usually natural products. The recent development of methods to synthesize hundreds of thousands of small molecules and to carry out large-scale automated screens holds the promise of identifying chemical inhibitors for virtually any biological process. In such approaches, large collections of small chemical compounds are simultaneously tested, either on living cells or in cell-free assays. Once an inhibitor is identified, it can be used as a probe to identify, through affinity chromatography or other means, the protein to which the inhibitor binds and, if disruption of protein function is therapeutic, as a drug in and of itself.

Protein Structure Can Be Determined Using X-Ray Diffraction. The main technique that has been used to discover the three-dimensional structure of molecules, including proteins, at atomic resolution is x-ray crystallography. X-rays, like light, are a form of electromagnetic radiation, but they have a much shorter wavelength, typically around 0.1 nm (the diameter of a hydrogen atom). If a narrow parallel beam of x-rays is directed at a sample of a pure protein, most of the x-rays pass straight through it. A small fraction, however, are scattered by the atoms in the sample. If the sample is a well-ordered crystal, the scattered waves reinforce one another at certain points and appear as diffraction spots when recorded by a suitable detector.

The position and intensity of each spot in the x-ray diffraction pattern contain information about the locations of the atoms in the crystal that gave rise to it. Deducing the three-dimensional structure of a large molecule from the diffraction pattern of its crystal is a complex task. But in recent years x-ray diffraction analysis has become increasingly automated, and now the slowest step is likely to be the generation of suitable protein crystals. This step requires large amounts of very pure protein and often involves years of trial and error to discover the proper crystallization conditions; the pace has somewhat accelerated with the use of recombinant DNA techniques to produce pure proteins and computerized techniques to test large numbers of crystallization conditions. Analysis of the resulting diffraction pattern produces a complex three dimensional electron-density map. Interpreting this map—translating its contours into a three-dimensional structure—is a complicated procedure that requires knowledge of the amino acid sequence of the protein. Largely by trial and error, the sequence and the electron-density map are correlated by computer to give the best possible fit. The reliability of the final atomic model depends on the resolution of the original crystallographic data: 0.5 nm resolution might produce a low-resolution map of the polypeptide backbone, whereas a resolution of 0.15 nm allows all of the nonhydrogen atoms in the molecule to be reliably positioned.

A complete atomic model is often too complex to appreciate directly, but simplified versions that show a protein's essential structural features can be readily derived from it. The three-dimensional structures of about 20,000 different proteins have now been determined by x-ray crystallography or by NMR spectroscopy—enough to begin to see families of common structures emerging. These structures or protein folds often seem to be more conserved in evolution than are the amino acid sequences that form the α helices and β strands themselves.

NMR Can Be Used to Determine Protein Structure in Solution. Nuclear magnetic resonance (NMR) spectroscopy has been widely used for many years to analyze the structure of small molecules. This technique is now also increasingly applied to the study of small proteins or protein domains. Unlike x-ray crystallography, NMR does not depend on having a crystalline sample. It simply requires a small volume of concentrated protein solution that is placed in a strong magnetic field; indeed, it is the main technique that yields detailed evidence about the three-dimensional structure of molecules in solution. Certain atomic nuclei, particularly hydrogen nuclei, have a magnetic moment or spin: that is, they have an intrinsic magnetization, like a bar magnet. The spin aligns along the strong magnetic field, but it can be changed to a misaligned, excited state in response to applied radiofrequency (RF) pulses of electromagnetic radiation. When the excited hydrogen nuclei return to their aligned state, they emit RF radiation, which can be measured and displayed as a spectrum. The nature of the emitted radiation depends on the environment of each hydrogen nucleus, and if one nucleus is excited, it influences the absorption and emission of radiation by other nuclei that lie close to it. It is consequently possible, by an ingenious elaboration of the basic NMR technique known as two-dimensional NMR, to distinguish the signals from hydrogen nuclei in different amino acid residues, and to identify and measure the small shifts in these signals that occur when these hydrogen nuclei lie close enough together to interact. Because the size of such a shift reveals the distance between the interacting pair of hydrogen atoms, NMR can provide information about the distances between the parts of the protein molecule. By combining this information with a knowledge of the amino acid sequence, it is possible in principle to compute the three-dimensional structure of the protein.

For technical reasons the structure of small proteins of about 20,000 daltons or less can be most readily determined by NMR spectroscopy. Resolution decreases as the size of a macromolecule increases. But recent technical advances have now pushed the limit to about 100,000 daltons, thereby making the majority of proteins accessible for structural analysis by NMR.

Protein Sequence and Structure Provide Clues About Protein Function. Having discussed methods for purifying and analyzing proteins, we now turn to a common situation in cell and molecular biology: an investigator has identified a gene important for a biological process but has no direct knowledge of the biochemical properties of its protein product. Thanks to the proliferation of protein and nucleic acid sequences that are catalogued in genome databases, the function of a gene—and its encoded protein—can conceivably be predicted by simply comparing its sequence with those of previously characterized genes. Because amino acid sequence determines protein structure, and structure dictates biochemical function, proteins that share a similar amino acid sequence usually have the same structure and usually perform similar biochemical functions, even when they are found in distantly related organisms. In modern cell biology, the study of a newly discovered protein usually begins with a search for previously characterized proteins that are similar in their amino acid sequences.

Searching a collection of known sequences for similar genes or proteins is typically done over the World Wide Web, and it conventionally involves selecting a database and entering the desired sequence. A sequence alignment program—the most popular are BLAST and FASTA—scans the database for similar sequences by sliding the submitted sequence along the archived sequences until a cluster of residues falls into full or partial alignment. The results of even a complex search—which can be performed on either a nucleotide or an amino acid sequence—are returned within time. Such comparisons can predict the functions of individual proteins, families of proteins, or even much of the protein complement of a newly sequenced organism.

Many proteins that adopt the same conformation and have related functions are too distantly related to be identified as clearly similar from a comparison of their amino acid sequences alone. Thus, an ability to reliably predict the three dimensional structure of a protein from its amino acid sequence would improve our ability to infer protein function from the sequence information in genomic databases. In recent years, major progress has been made in predicting the precise structure of a protein. These predictions are based, in part, on our knowledge of tens of thousands of protein structures that have already been determined by x-ray crystallography and NMR spectroscopy and, in part, on computations using our knowledge of the physical forces acting on the atoms. However, it remains a substantial and important challenge to predict the structures of proteins that are large or have multiple domains, or to predict structures at the very high levels of resolution needed to assist in computer-based drug discovery.

Sequence databases can be searched (or two or more sequences can be aligned) to find similar amino acid or nucleic acid sequences. For example, a BLAST search for proteins similar to the human cell-cycle regulatory protein Cdc2 (Query) locates maize Cdc2 (Sbjct), which is 68% identical (and 82% similar) to human Cdc2 in its amino acid sequence. The alignment begins at residue 57 of the Query protein, suggesting that the human protein has an N-terminal region that is absent from the maize protein. The results of the BLAST search indicate differences in sequence as well as similarities, and when the two amino acid sequences are identical as well as when conservative amino acids are substituted. Here, only one small gap needs to be introduced, at position 194 in the Query sequence, to align the two sequences maximally. The alignment score (Score), which is expressed in two different types of units, takes into account penalties for substitutions and gaps; the higher the alignment score, the better the match. The significance of the alignment is reflected in the Expectation (E) value, which specifies how often a match this good would be expected to occur by chance. The lower the E value, the more significant the match; the very low value in this instance e-111 indicates certain significance. E values much higher than 0.1 are unlikely to reflect true relatedness. For example, an E value of 0.1 means there is a 1 in 10 likelihood that such a match would arise solely by chance. Protein sequence alignments use standard substitution matrices, for example, the BLOSUM62 matrix, that take into account matches and mismatches of different types (such as a proline to valine, or isoleucine to leucine) based on their different physicochemical and evolutionary properties. Amino acids that are physicochemically similar to one another are determined by their side chains. The common amino acids are grouped according to whether their side chains are acidic, basic, uncharged polar, or nonpolar. Of the 20 amino acids found in proteins, there are equal numbers of polar and non-polar side chains. However, some side chains considered polar are large enough to have some non-polar properties, e.g., Tyr, Thr, Arg, and Lys. Here is the list of amino acids, with their 3 letter abbreviation, 1 letter abbreviation, and grouping by side chain.

| Amino acid | 3 letter name | 1 letter name | Side chain |
|---|---|---|---|
| Aspartic acid | Asp | D | Negative (polar) |
| Glutamic acid | Glu | E | Negative (polar) |
| Arginine | Arg | R | Positive (polar) |
| Lysine | Lys | K | Positive (polar) |
| Histidine | His | H | Positive (polar) |
| Asparagine | Asn | N | Uncharged polar |
| Glutamine | Gln | Q | Uncharged polar |
| Serine | Ser | S | Uncharged polar |
| Threonine | Thr | T | Uncharged polar |
| Tyrosine | Tyr | Y | Uncharged polar |
| Alanine | Ala | A | Nonpolar |
| Glycine | Gly | G | Nonpolar |
| Valine | Val | V | Nonpolar |
| Leucine | Leu | L | Nonpolar |
| Isoleucine | Ile | I | Nonpolar |
| Proline | Pro | P | Nonpolar |
| Phenylalanine | Phe | F | Nonpolar |
| Methionine | Met | M | Nonpolar |
| Tryptophan | Trp | W | Nonpolar |
| Cysteine | Cys | C | Nonpolar |

Generally speaking, one requires a 30% identity in sequence to consider that two polypeptides match. While finding similar sequences and structures for a new protein will provide many clues about its function, it may be necessary to test these insights through direct experimentation. However, the clues generated from sequence comparisons traditionally point the investigator in the correct experimental direction. The use of sequence alignments has therefore become one of the choicest strategies in modern cell biology.

Analyzing and Manipulating DNA. Technical breakthroughs in genetic engineering—the ability to manipulate DNA with precision in a test tube or an organism—have had a dramatic impact on all aspects of cell biology by facilitating the study of cells and their macromolecules in previously unimagined ways. Recombinant DNA technology comprises a mixture of techniques, some newly developed and some borrowed from other fields. Central to the technology are the following key techniques: 1. Cleavage of DNA at specific sites by restriction nucleases, which greatly facilitates the isolation and manipulation of individual genes. 2. DNA ligation, which makes it possible to design and construct DNA molecules that are not found in nature. 3. DNA cloning through the use of either cloning vectors or the polymerase chain reaction, in which a portion of DNA is repeatedly copied to generate many billions of identical molecules. 4. Nucleic acid hybridization, which makes it possible to find a specific sequence of DNA or RNA with great accuracy and sensitivity on the basis of its ability to selectively bind a complementary nucleic acid sequence. 5. Determination of the sequence of nucleotides of any DNA (even entire genomes), making it possible to identify genes and to deduce the amino acid sequence of the proteins they encode. 6. Simultaneous monitoring of the level of mRNA produced by genes in a cell using nucleic acid microarrays, in which tens of thousands of hybridization reactions take place simultaneously.

Restriction Nucleases Cut Large DNA Molecules into Fragments. Unlike a protein, a gene does not exist as a discrete entity in cells, but rather as a small region of a much longer DNA molecule. Although the DNA molecules in a cell can be randomly broken into small pieces by mechanical force, a fragment containing a single gene in a mammalian genome would still be only one among a hundred thousand or more DNA fragments, indistinguishable in their average size. How could such a gene be purified? Because all DNA molecules consist of an approximately equal mixture of the same four nucleotides, they cannot be readily separated, as proteins can, on the basis of their different charges and binding properties.

The solution to all of these problems began to emerge with the discovery of restriction nucleases. These enzymes, which can be purified from bacteria, cut the DNA double helix at specific sites defined by the local nucleotide sequence, thereby cleaving a long double-stranded DNA molecule into fragments of strictly defined sizes. Different restriction nucleases have different sequence specificities, and it is straightforward to find an enzyme that can create a DNA fragment that includes a particular gene. The size of the DNA fragment can then be used as a basis for partial purification of the gene from a mixture.

Different species of bacteria make different restriction nucleases, which protect them from viruses by degrading incoming viral DNA. Each bacterial nuclease recognizes a specific sequence of four to eight nucleotides in DNA. These sequences, where they occur in the genome of the bacterium itself, are protected from cleavage by methylation at an A or a C nucleotide; the sequences in foreign DNA are generally not methylated and so are cleaved by the restriction nucleases. Large numbers restriction nucleases have been purified from various species of bacteria; several hundred, most of which recognize different nucleotide sequences, are now available commercially.

Some restriction nucleases produce staggered cuts, which leave short single stranded tails at the two ends of each fragment. Ends of this type are known as cohesive ends, as each tail can form complementary base pairs with the tail at any other end produced by the same enzyme. The cohesive ends generated by restriction enzymes allow any two DNA fragments to be easily joined together, as long as the fragments were generated with the same restriction nuclease (or with another nuclease that produces the same cohesive ends). DNA molecules produced by splicing together two or more DNA fragments are called recombinant DNA molecules.

Gel Electrophoresis Separates DNA Molecules of Different Sizes. The same types of gel electrophoresis methods that have proved so useful in the analysis of proteins can determine the length and purity of DNA molecules. The procedure is actually simpler than for proteins: because each nucleotide in a nucleic acid molecule already carries a single negative charge (on the phosphate group), there is no need to add the negatively charged detergent SDS that is required to make protein molecules move uniformly toward the positive electrode. For DNA fragments less than 500 nucleotides long, specially designed polyacrylamide gels allow the separation of molecules that differ in length by as little as a single nucleotide. The pores in polyacrylamide gels, however, are too small to permit very large DNA molecules to pass; to separate these by size, the much more porous gels formed by dilute solutions of agarose (a polysaccharide isolated from seaweed) are used. These DNA separation methods are widely used for both analytical and preparative purposes.

A variation of agarose-gel electrophoresis, called pulsed-field gel electrophoresis, makes it possible to separate even extremely long DNA molecules. Ordinary gel electrophoresis fails to separate such molecules because the steady electric field stretches them out so that they travel end-first through the gel in snakelike configurations at a rate that is independent of their length. In pulsed-field gel electrophoresis, by contrast, the direction of the electric field changes periodically, which forces the molecules to reorient before continuing to move snakelike through the gel. This reorientation takes much more time for larger molecules, so that longer molecules move more slowly than shorter ones. As a consequence, even entire bacterial or yeast chromosomes separate into discrete bands in pulsed-field gels and so can be sorted and identified on the basis of their size. Although a typical mammalian chromosome of 10̂8 base pairs is too large to be sorted even in this way, large segments of these chromosomes are readily separated and identified if the chromosomal DNA is first cut with a restriction nuclease selected to recognize sequences that occur only rarely (once every 10,000 or more nucleotide pairs).

The DNA bands on agarose or polyacrylamide gels are invisible unless the DNA is labeled or stained in some way. One sensitive method of staining DNA is to expose it to the dye ethidium bromide, which fluoresces under ultraviolet light when it is bound to DNA. An even more sensitive detection method incorporates a radioisotope into the DNA molecules before electrophoresis; 32P is often used as it can be incorporated into DNA phosphates and emits an energetic βparticle that is easily detected by autoradiography.

Purified DNA Molecules Can Be Specifically Labeled with Radioisotopes or Chemical Markers in vitro. Two procedures are widely used to label isolated DNA molecules. In the first method, a DNA polymerase copies the DNA in the presence of nucleotides that are either radioactive (usually labeled with 32P) or chemically tagged. In this way, “DNA probes” containing many labeled nucleotides can be produced for nucleic acid hybridization reactions. The second procedure uses the bacteriophage enzyme polynucleotide kinase to transfer a single 32P-labeled phosphate from ATP to the 5′ end of each DNA chain. Because only one 32P atom is incorporated by the kinase into each DNA strand, the DNA molecules labeled in this way are often not radioactive enough to be used as DNA probes; because they are labeled at only one end, however, they have been invaluable for other applications, including DNA footprinting. Radioactive labeling methods are being replaced by labeling with molecules that can be detected chemically or through fluorescence. To produce such nonradioactive DNA molecules, specially modified nucleotide precursors are used. A DNA molecule made in this way is allowed to bind to its complementary DNA sequence by hybridization, and is then detected with an antibody (or other ligand) that specifically recognizes its modified side chain.

Nucleic Acid Hybridization Reactions Provide a Sensitive Way to Detect Specific Nucleotide Sequences. When an aqueous solution of DNA is heated at 100° C. or exposed to a very high pH (pH>13), the complementary base pairs that normally hold the two strands of the double helix together are disrupted and the double helix rapidly dissociates into two single strands. This process, called DNA denaturation, was for many years thought to be irreversible. It was discovered, however, that complementary single strands of DNA readily re-form double helices by a process called hybridization (also called DNA renaturation) if they are kept for a prolonged period at 65° C. Similar hybridization reactions can occur between any two single-stranded nucleic acid chains (DNA/DNA, RNA/RNA, or RNA/DNA), provided that they have complementary nucleotide sequences. These specific hybridization reactions are widely used to detect and characterize specific nucleotide sequences in both RNA and DNA molecules. Single-stranded DNA molecules used to detect complementary sequences are known as probes; these molecules, which carry radioactive or chemical markers to facilitate their detection, can range from fifteen to thousands of nucleotides long.

Hybridization reactions using DNA probes are so sensitive and selective that they can detect complementary sequences present at a concentration as low as one molecule per cell. It is thus possible to determine how many copies of any DNA sequence are present in a particular DNA sample. The same technique can be used to search for similar but nonidentical genes. To find a gene of interest in an organism whose genome has not yet been sequenced, for example, a portion of a known gene can be used as a probe.

Stringent versus nonstringent hybridization conditions tell sequences apart. To use a DNA probe to find an identical match, stringent hybridization conditions are used; the reaction temperature is kept just a few degrees below that at which a perfect DNA helix denatures in the solvent used (its melting temperature), so that all imperfect helices formed are unstable. Lowering the salt concentration lowers the melting point, as does the addition of formamide. As an example, hybridization is in 50% formamide at 42° C. When a DNA probe is being used to find DNAs with similar, as well as identical, sequences, less stringent conditions are used; hybridization is performed at a lower temperature, which allows even imperfectly paired double helices to form. Continuing with this example, hybridization is in 50% formamide at 35° C. Only the lower temperature hybridization conditions can be used to search for genes that are nonidentical but similar. As another example, high stringency hybridization is in 6×SSC, 0.2% SDS, 1×Denhardt's blocking solution, or 1% w/v milk, 10-50 ng/ml probe (denatured), 65° C. incubation, with agitation, for 18-24 hours. Following a period of hybridization, it is necessary to wash off the probe that is loosely bound to the target (i.e., nonspecifically bound). Continuing with this example, high stringency wash is at 25° C. in decreasing salt concentrations (i.e., 3×SSC/0.2% SDS, then 1×SSC/0.2% SDS).

Alternatively, DNA probes can be used in hybridization reactions with RNA rather than DNA to find out whether a cell is expressing a given gene. In this case a DNA probe that contains part of the gene's sequence is hybridized with RNA purified from the cell in question to see whether the RNA includes nucleotide sequences matching the probe DNA and, if so, in what quantities. In somewhat more elaborate procedures, the DNA probe is treated with specific nucleases after the hybridization is complete, to determine the exact regions of the DNA probe that have paired with the RNA molecules. One can thereby determine the start and stop sites for RNA transcription, as well as the precise boundaries of the intron and exon sequences in a gene.

Today, the positions of intron/exon boundaries are usually determined by sequencing the complementary DNA (cDNA) sequences that represent the mRNAs expressed in a cell and comparing them with the nucleotide sequence of the genome. We describe later how cDNAs are prepared from mRNAs. The hybridization of DNA probes to RNAs allows one to determine whether or not a particular gene is being transcribed; moreover, when the expression of a gene changes, one can determine whether the change is due to transcriptional or posttranscriptional controls. These tests of gene expression were initially performed with one DNA probe at a time. DNA microarrays now allow the simultaneous monitoring of hundreds or thousands of genes at a time. Hybridization methods are still in wide use in cell biology today.

Northern and Southern Blotting Facilitate Hybridization with Electrophoretically Separated Nucleic Acid Molecules. Specific RNA or DNA molecules are detected by gel-transfer hybridization in a method called Southern blotting (named after its inventor) or Northern blotting (named with reference to Southern blotting). To start, one collects tissue from a source and disrupts the cells in a strong detergent to inactivate nucleases that might otherwise degrade the nucleic acids. Next, one separates the RNA and DNA from all of the other cell components: the proteins present are completely denatured and removed by repeated extractions with phenol—a potent organic solvent that is partly miscible with water; the nucleic acids, which remain in the aqueous phase, are then precipitated with alcohol to separate them from the small molecules of the cell. Then one separates the DNA from the RNA by their different solubilities in alcohols and degrades any contaminating nucleic acid of the unwanted type by treatment with a highly specific enzyme—either an RNase or a DNase. The mRNAs are typically separated from bulk RNA by retention on a chromatography column that specifically binds the poly-A tails of mRNAs.

In this example, the DNA probe is detected by its radioactivity. DNA probes detected by chemical or fluorescence methods are also widely used. First, a mixture of either single-stranded RNA molecules (Northern blotting) or the double-stranded DNA fragments created by restriction nuclease treatment (Southern blotting) is separated according to length by electrophoresis. Next, a sheet of nitrocellulose or nylon paper is laid over the gel, and the separated RNA or DNA fragments are transferred to the sheet by blotting. Then, the nitrocellulose sheet is carefully peeled off the gel. Next, the sheet containing the bound nucleic acids is placed in a sealed plastic bag together with a buffered salt solution containing a radioactively labeled DNA probe. The sheet is exposed to a labeled DNA probe for a prolonged period under conditions favoring hybridization. Last, the sheet is removed from the bag and washed thoroughly, so that only probe molecules that have hybridized to the RNA or DNA immobilized on the paper remain attached. After autoradiography, the DNA that has hybridized to the labeled probe shows up as bands on the autoradiograph. For Southern blotting, the strands of the double-stranded DNA molecules on the paper must be separated before the hybridization process; this is done by exposing the DNA to alkaline denaturing conditions after the gel has been run.

Genes Can Be Cloned Using DNA Libraries. Genes can be cloned using DNA libraries. Almost any DNA fragment can be cloned. In molecular biology, the term DNA cloning is used in two senses. In one sense, it literally refers to the act of making many identical copies of a DNA molecule—the amplification of a particular DNA sequence. However, the term also describes the isolation of a particular stretch of DNA (often a particular gene) from the rest of a cell's DNA, because this isolation is greatly facilitated by making many identical copies of the DNA of interest. In both cases, cloning refers to the act of making many genetically identical copies.

DNA cloning in its most general sense can be accomplished in several ways. The simplest involves inserting a particular fragment of DNA into the purified DNA genome of a self-replicating genetic element—generally a virus or a plasmid. A DNA fragment containing a human gene, for example, can be joined in a test tube to the chromosome of a bacterial virus, and the new recombinant DNA molecule can then be introduced into a bacterial cell, where the inserted DNA fragment will be replicated along with the DNA of the virus. Starting with only one such recombinant DNA molecule that infects a single cell, the normal replication mechanisms of the virus can produce more than 10 to the power of 12 identical virus DNA molecules in a single day, thereby amplifying the amount of the inserted human DNA fragment by the same factor. A virus or plasmid used in this way is known as a cloning vector, and the DNA propagated by insertion into it is said to have been cloned. To isolate a specific gene, one begins by constructing a DNA library—a comprehensive collection of cloned DNA fragments from a cell, tissue, or organism. This library includes (one hopes) at least one fragment that contains the gene of interest. Libraries can be constructed with either a virus or a plasmid vector and are generally housed in a population of bacterial cells. The principles underlying the methods used for cloning genes are the same for either type of cloning vector, although the details may differ. Today, most cloning is performed with plasmid vectors.

The plasmid vectors most widely used for gene cloning are small circular molecules of double-stranded DNA derived from larger plasmids that occur naturally in bacterial cells. They generally account for only a minor fraction of the total host bacterial cell DNA, but they can easily be separated owing to their small size from chromosomal DNA molecules, which are large and precipitate as a pellet upon centrifugation. For use as cloning vectors, the purified plasmid DNA circles are first cut with a restriction nuclease to create linear DNA molecules. The genomic DNA to be used in constructing the library is cut with the same restriction nuclease, and the resulting restriction fragments (including those containing the gene to be cloned) are then added to the cut plasmids and annealed via their cohesive ends to form recombinant DNA circles. These recombinant molecules containing foreign DNA inserts are then covalently sealed with the enzyme DNA ligase.

In the next step in preparing the library, the recombinant DNA circles are introduced into bacterial cells that have been made transiently permeable to DNA. These bacterial cells are now said to be transfected with the plasmids. As the cells grow and divide, doubling in number every 30 minutes, the recombinant plasmids also replicate to produce an enormous number of copies of DNA circles containing the foreign DNA. Many bacterial plasmids carry genes for antibiotic resistance, a property that can be exploited to select those cells that have been successfully transfected; if the bacteria are grown in the presence of the antibiotic, only cells containing plasmids will survive. Each original bacterial cell that was initially transfected contains, in general, a different foreign DNA insert; this insert is inherited by all of the progeny cells of that bacterium, which together form a small colony in a culture dish.

For many years, plasmids were used to clone fragments of DNA of 1000 to 30,000 nucleotide pairs. Larger DNA fragments are more difficult to handle and were harder to clone. Today, new plasmid vectors based on the naturally occurring F plasmid of E. coli are used to clone DNA fragments of 300,000 to 1 million nucleotide pairs. Unlike smaller bacterial plasmids, the F plasmid—and its derivative, the bacterial artificial chromosome (BAC)—is present in only one or two copies per E. coli cell. The fact that BACs are kept in such low numbers in bacterial cells may contribute to their ability to maintain large cloned DNA sequences stably: with only a few BACs present, it is less likely that the cloned DNA fragments will become scrambled by recombination with sequences carried on other copies of the plasmid. Because of their stability, ability to accept large DNA inserts, and ease of handling, BACs are now the preferred vector for building DNA libraries of complex organisms—including those representing the human genome.

Two Types of DNA Libraries Serve Different Purposes. Cleaving the entire genome of a cell with a specific restriction nuclease and cloning each fragment as just described produces a very large number of DNA fragments—on the order of a million for a mammalian genome. The fragments are distributed among millions of different colonies of transfected bacterial cells. Each of the colonies is composed of a clone of cells derived from a single ancestor cell, and therefore harbors many copies of a particular stretch of the fragmented genome. Such a plasmid is said to contain a genomic DNA clone, and the entire collection of plasmids is called a genomic DNA library. But because the genomic DNA is cut into fragments at random, only some fragments contain genes. Many of the genomic DNA clones obtained from the DNA of a higher eukaryotic cell contain only noncoding DNA, which makes up most of the DNA in such genomes. An alternative strategy is to begin the cloning process by selecting only those DNA sequences that are transcribed into mRNA and thus are presumed to correspond to protein-encoding genes. This is done by extracting the mRNA from cells and then making a DNA copy of each mRNA molecule present—a so-called complementary DNA, or cDNA. The copying reaction is catalyzed by the reverse transcriptase enzyme of retroviruses, which synthesizes a complementary DNA chain on an RNA template. The single-stranded cDNA molecules synthesized by the reverse transcriptase are converted into double-stranded cDNA molecules by DNA polymerase, and these molecules are inserted into a plasmid or virus vector and cloned. Each clone obtained in this way is called a cDNA clone, and the entire collection of clones derived from one mRNA preparation constitutes a cDNA library.

cDNA Clones Contain Uninterrupted Coding Sequences. There are some important differences between genomic DNA clones and cDNA clones. Genomic clones represent a random sample of all the DNA sequences in an organism and, with very rare exceptions, are the same regardless of the cell type used to prepare them. By contrast, cDNA clones contain only those regions of the genome that have been transcribed into mRNA. Because the cells of different tissue types produce distinct sets of mRNA molecules, a distinct cDNA library is obtained for each type of cell used to prepare the library.

The most important advantage of cDNA clones is that they contain the uninterrupted coding sequence of a gene. Eukaryotic genes usually consist of short coding sequences of DNA (exons) separated by much longer noncoding sequences (introns); the production of mRNA entails the removal of the noncoding sequences from the initial RNA transcript and the splicing together of the coding sequences. Bacterial cells will not make these modifications to the RNA produced from a gene of a higher eukaryotic cell. Thus, when the aim of the cloning is either to deduce the amino acid sequence of the protein from the DNA sequence or to produce the protein in bulk by expressing the cloned gene in a bacterial cell, it is much preferable to start with cDNA. cDNA libraries have the additional advantage of representing alternatively spliced mRNAs produced rom a given cell or tissue. Genomic and cDNA libraries are widely shared among investigators and, today, many such libraries are also available from commercial sources.

Genes Can Be Selectively Amplified by PCR. Now that so many genome sequences are available, genes can be cloned directly without the need to first construct DNA libraries. A technique called polymerase chain reaction (PCR) makes this rapid cloning possible. Starting with an entire genome, PCR allows the DNA from a selected region to be amplified several billionfold, effectively “purifying” this DNA away from the remainder of the genome. To begin, a pair of DNA oligonucleotides, chosen to flank the desired nucleotide sequence of the gene, are synthesized by chemical methods. These oligonucleotides are then used to prime DNA synthesis on single strands generated by heating the DNA from the entire genome. The newly synthesized DNA is produced in a reaction catalyzed in vitro by a purified DNA polymerase, and the primers remain at 20 the 5′ ends of the final DNA fragments that are made.

Nothing special is produced in the first cycle of DNA synthesis; the power of the PCR method is revealed only after repeated rounds of DNA synthesis. Every cycle doubles the amount of DNA synthesized in the previous cycle. Because each cycle requires a brief heat treatment to separate the two strands of the template DNA double helix, the technique requires the use of a special DNA polymerase, isolated from a thermophilic bacterium, that is stable at much higher temperatures than normal so that it is not denatured by the repeated heat treatments. With each round of DNA synthesis, the newly generated fragments serve as templates in their turn, and within a few cycles the predominant product is a single species of DNA fragment whose length corresponds to the distance between the two original primers.

In practice, effective DNA amplification requires 20-30 reaction cycles, with the products of each cycle serving as the DNA templates for the next—hence the term polymerase “chain reaction.” A single cycle requires only about 5 minutes, and the entire procedure can be easily automated. PCR thereby makes possible the “cell-free molecular cloning” of a DNA fragment in a few hours, compared with the several days for standard cloning procedures. This technique is now used routinely to clone DNA from genes of interest directly—starting either from genomic DNA or from mRNA isolated from cells.

The PCR method is extremely sensitive; it can detect a single DNA molecule in a sample. Trace amounts of RNA can be analyzed in the same way by first transcribing them into DNA with reverse transcriptase. The PCR cloning technique has largely replaced Southern blotting for the diagnosis of genetic diseases and for the detection of low levels of viral infection.

Expression of Individual Genes Can Be Measured Using Quantitative Real-Time PCR (qPCR). It is often desirable to quantitate gene expression by directly measuring mRNA levels in cells. Although Northern blots can be adapted to this purpose, a more accurate method is based on the principles of PCR. This method, called quantitative RT-PCR (reverse transcription-polymerase chain reaction), begins with the total population of mRNA molecules purified from a tissue or a cell culture. It is important that no DNA be present in the preparation; it must be purified away or enzymatically degraded. Two DNA primers that specifically match the gene of interest are added, along with reverse transcriptase, DNA polymerase, and the four deoxynucleoside triphosphates needed for DNA synthesis. The first round of synthesis is the reverse transcription of the mRNA into DNA using one of the primers. Next, a series of heating and cooling cycles allows the amplification of that DNA strand by conventional PCR. The quantitative part of this method relies on a direct relationship between the rate at which the PCR product is generated and the original concentration of the mRNA species of interest. By adding chemical dyes to the PCR reaction that fluoresce only when bound to double-stranded DNA, a simple fluorescence measurement can be used to track the progress of the reaction and thereby accurately deduce the starting concentration of the mRNA that is amplified. Although it seems complicated, this quantitative RT-PCR technique (sometimes called real time PCR) is straightforward to perform in the laboratory; it has displaced Northern blotting as the method of choice for quantifying mRNA levels from any given gene.

Cells Can Be Used As Factories to Produce Specific Proteins. The vast majority of the thousands of different proteins in a cell, including many with crucially important functions, are present in very small amounts. In the past, for most of them, it has been extremely difficult, if not impossible, to obtain more than a few micrograms of pure material. One of the most important contributions of DNA cloning and genetic engineering to cell biology is that they have made it possible to produce almost any of the cell's proteins in a nearly unlimited amount. Large amounts of the desired protein are produced in living cells by using expression vectors. These are generally plasmids that have been designed to produce a large amount of a stable mRNA that can be efficiently translated into protein in the transfected bacterial, yeast, insect, or mammalian cell. A plasmid vector is engineered to contain a highly active promoter, which causes unusually large amounts of mRNA to be produced from an adjacent protein-coding gene inserted into the plasmid vector. Depending on the characteristics of the cloning vector, the plasmid is introduced into bacterial, yeast, insect, or mammalian cells, where the inserted gene is efficiently transcribed and translated into protein.

Because the desired protein made from an expression vector is produced inside a cell, it must be purified away from the host-cell proteins by chromatography after cell lysis; but because it is a plentiful species in the cell lysate (often 1-10% of the total cell protein), the purification is usually easy to accomplish in only a few steps. In order to purify a protein, it first must be extracted from inside the cell, unless it is secreted into the medium. The cells are typically homogenized to produce a homogenate or slurry. The homogenate is typically fractionated into different components by centrifugation. After centrifugation, proteins are often separated by chromatography. Secreted, soluble proteins are isolated from the supernatants of infected cells after pelleting the cells by centrifugation and do not require cell lysis. A variety of expression vectors are available, each engineered to function in the type of cell in which the protein is to be made. In this way, cells can be induced to make vast quantities of proteins useful for medical purposes or to be studied for structure and function.

Genes Can Be Engineered by Site-Directed Mutagenesis. A technique called site-directed mutagenesis changes selected amino acids in a protein. To begin, a recombinant plasmid containing a gene insert is separated into its two DNA strands. A synthetic oligonucleotide primer corresponding to part of the gene sequence but containing a single altered nucleotide at a predetermined point is added to the single-stranded DNA under conditions that permit imperfect DNA hybridization. The primer hybridizes to the DNA, forming a single mismatched nucleotide pair. The recombinant plasmid is made double-stranded by in vitro DNA synthesis (starting from the primer) followed by sealing by DNA ligase. The double stranded DNA is introduced into a cell, where it is replicated. Replication using one strand of the template produces a normal DNA molecule, but replication using the other strand (the one that contains the primer) produces a DNA molecule carrying the desired mutation. Only half of the progeny cells will end up with a plasmid that contains the desired mutant gene. However, a progeny cell that contains the mutated gene can be identified, separated from other cells, and cultured to produce a pure population of cells, all of which carry the mutated gene. With an oligonucleotide of the appropriate sequence, more than one amino acid substitution can be made at a time, or one or more amino acids can be inserted or deleted. It is also possible to create a site-directed mutation by using the appropriate oligonucleotides and PCR (instead of plasmid replication) to amplify the mutated gene.

Proteins and Nucleic Acids Can Be Synthesized Directly By Chemical Reactions. Chemical reactions have been devised to synthesize directly specific sequences of amino acids or nucleic acids. These methodologies provide direct sources of biological molecules and do not rely on any cells or enzymes. Chemical synthesis is the method of choice for obtaining nucleic acids in the range of 100 nucleotides or fewer, which, under the basic concept of de novo gene synthesis, may be assembled into larger constructs using some form of polymerase chain assembly or ligase chain reaction approach. Chemical synthesis is also routinely used to produce specific peptides that, when chemically coupled to other proteins, are used to generate antibodies against the peptide.

DNA Can Be Rapidly Sequenced. The dideoxy method for sequencing DNA is based on in vitro DNA synthesis performed in the presence of chain-terminating dideoxyribonucleoside triphosphates. This method relies on the use of dideoxyribonucleoside triphosphates, derivatives of the normal deoxyribonucleoside triphosphates that lack the 3′ hydroxyl group. Purified DNA is synthesized in vitro in a mixture that contains single-stranded molecules of the DNA to be sequenced, the enzyme DNA polymerase, a short primer DNA to enable the polymerase to start DNA synthesis, and the four deoxyribonucleoside triphosphates (dATP, dCTP, dGTP, dTTP). If a dideoxyribonucleotide analog of one of these nucleotides is also present in the nucleotide mixture, it can become incorporated into a growing DNA chain. Because this chain now lacks a 3′ OH group, the addition of the next nucleotide is blocked, and the DNA chain terminates at that point. As an example, if a small amount of dideoxy ATP (ddATP) is added to the nucleotide mixture, it competes with an excess of the normal deoxyATP (dATP), so that ddATP is occasionally incorporated, at random, into a growing DNA strand. This reaction mixture will eventually produce a set of DNAs of different lengths complementary to the template DNA that is being sequenced and terminating at each of the different As. The exact lengths of the DNA synthesis products can then be used to determine the position of each A in the growing chain. To determine the complete sequence of a DNA fragment, the doublestranded DNA is first separated into its single strands and one of the strands is used as the template for sequencing. Four different chain-terminating dideoxyribonucleoside triphosphates (ddATP, ddCTP, ddGTP, ddTTP) are used in four separate DNA synthesis reactions on copies of the same single-stranded DNA template. Each reaction produces a set of DNA copies that terminate at different points in the sequence. The products of these four reactions are separated by electrophoresis in four parallel lanes of a polyacrylamide gel. The newly synthesized fragments are detected by a label (either radioactive or fluorescent) that has been incorporated either into the primer or into one of the deoxyribonucleoside triphosphates used to extend the DNA chain. In each lane, the bands represent fragments that have terminated at a given nucleotide but at different positions in the DNA. By reading off the bands in order, starting at the bottom of the gel and working across all lanes, the DNA sequence of the newly synthesized strand can be determined. This sequence is complementary to the template strand from the original double-stranded DNA molecule, and identical to a portion of the 5′-to-3′ strand.

Today, sequencing is often carried out by automated machines that use fluorescent dyes and laser scanners. The dideoxy reaction is also used here, but the ddNTPs used in the reaction are labeled with a fluorescent dye, and a different colored dye is used for each type of dideoxynucleotide. In this case, the four sequencing reactions can take place in the same test tube and can be placed in the same well during electrophoresis. The most recently developed sequencing machines carry out electrophoresis in gel-containing capillary tubes. During electrophoresis, the fragments migrate through the gel according to size, and the fluorescent dye on the DNA is activated by a laser beam and detected by an optical scanner. The results are printed as a set of peaks on a graph.

Nucleotide Sequences Are Used to Predict the Amino Acid Sequences of Proteins. Now that DNA sequencing is so rapid and reliable, it has become the preferred method for determining, indirectly, the amino acid sequences of most proteins. Given a nucleotide sequence that encodes a protein, the procedure is quite straightforward. Although in principle there are six different reading frames in which a DNA sequence can be translated into protein (three on each strand), the correct one is generally recognizable as the only one lacking frequent stop codons. A random sequence of nucleotides, read in frame, will encode a stop signal for protein synthesis about once every 20 amino acids. Nucleotide sequences that encode a stretch of amino acids much longer than this are candidates for presumptive exons, and they can be translated (by computer) into amino acid sequences and checked against databases for similarities to known proteins from other organisms. If necessary, a limited amount of amino acid sequence can then be determined from the purified protein to confirm the sequence predicted from the DNA.

The problem comes, however, in determining which nucleotide sequences—within a whole genome—represent genes that encode proteins. Identifying genes is easiest when the DNA sequence is from a bacterial or archaeal chromosome, which lacks introns, or from a cDNA clone. The location of genes in these nucleotide sequences can be predicted by examining the DNA for certain distinctive features. Briefly, these genes that encode proteins are identified by searching the nucleotide sequence for open reading frames (ORFs) that begin with an initiation codon, usually ATG, and end with a termination codon, TAA, TAG, or TGA. To minimize errors, computers used to search for ORFs are often directed to count as genes only those sequences that are longer than, say, 100 codons in length. For more complex genomes, such as those of animals and plants, the presence of large introns embedded within the coding portion of genes complicates the process.

In many multicellular organisms, including humans, the average exon is only 150 nucleotides long. Thus one must also search for other features that signal the presence of a gene, for example, sequences that signal an intron/exon boundary or distinctive upstream regulatory regions. Recent efforts to solve the exon prediction problem have turned to artificial intelligence algorithms, in which the computer learns, based on known examples, what sets of features are most indicative of an exon boundary. A second major approach to identifying the coding regions in chromosomes is through the characterization of the nucleotide sequences of the detectable mRNAs (using the corresponding cDNAs). The mRNAs (and the cDNAs produced from them) lack introns, regulatory DNA sequences, and the nonessential “spacer” DNA that lies between genes. It is therefore useful to sequence large numbers of cDNAs to produce a very large database of the coding sequences of an organism. These sequences are then readily used to distinguish the exons from the introns in the long chromosomal DNA sequences that correspond to genes.

The Genomes of Many Organisms Have Been Fully Sequenced. Owing in large part to the automation of DNA sequencing, the genomes of many organisms have been fully sequenced; these include plant chloroplasts and animal mitochondria, large numbers of bacteria, and archaea, and many of the model organisms that are studied routinely in the laboratory, including many yeasts, a nematode worm, the fruit fly Drosophila, the model plant Arabidopsis, the mouse, dog, chimpanzee, and, last but not least, humans. Researchers have also deduced the complete DNA sequences for a wide variety of human pathogens. These include the bacteria that cause cholera, tuberculosis, syphilis, gonorrhea, Lyme disease, and stomach ulcers, as well as hundreds of viruses—including smallpox virus and Epstein-Barr virus (which causes infectious mononucleosis). Examination of the genomes of these pathogens provides clues about what makes them virulent and will also point the way to new and more effective treatments.

Haemophilus influenzae (a bacterium that can cause ear infections and meningitis in children) was the first organism to have its complete genome sequence—all 1.8 million nucleotide pairs—determined by the shotgun sequencing method, the most common strategy used today. In the shotgun method, long sequences of DNA are broken apart randomly into many shorter fragments. Each fragment is then sequenced and a computer is used to order these pieces into a whole chromosome or genome, using sequence overlap to guide the assembly. The shotgun method is the technique of choice for sequencing small genomes. Although larger, more repetitive genome sequences are more challenging to assemble, the shotgun method—in combination with the analysis of large DNA fragments cloned in bacterial artificial chromosomes—has played a key role in their sequencing as well. With new sequences appearing at a steadily accelerating pace in the scientific literature, comparison of the complete genome sequences of different organisms allows us to trace the evolutionary relationships among genes and organisms, and to discover genes and predict their functions. Assigning functions to genes often involves comparing their sequences with related sequences from model organisms that have been well characterized in the laboratory, such as the bacterium E. coli, the yeasts S. cerevisiae and S. pombe, the nematode worm C. elegans, and the fruit fly Drosophila.

Although the organisms whose genomes have been sequenced share many biochemical pathways and possess many proteins that are similar in their amino acid sequence or structure, the functions of a very large number of newly identified proteins remain unknown. Depending on the organism, some 15-40% of the proteins encoded by a sequenced genome do not resemble any protein that has been studied biochemically. This observation underscores a limitation of the emerging field of genomics: although comparative analysis of genomes reveals a great deal of information about the relationships between genes and organisms, it often does not provide immediate information about how these genes function, or what roles they have in the physiology of an organism. Further biochemical and genetic studies are required to determine how genes, and the proteins they produce, function in the context of living organisms.

Microarrays Monitor the Expression of Thousands of Genes at Once DNA microarrays have revolutionized the analysis of gene expression by monitoring the RNA products of thousands of genes at once. By examining the expression of so many genes simultaneously, we can now begin to identify and study the gene expression patterns that underlie cell physiology: we can see which genes are switched on (or off) as cells grow, divide, differentiate, or respond to hormones or to toxins.

DNA microarrays are little more than glass microscope slides studded with a large number of DNA fragments, each containing a nucleotide sequence that serves as a probe for a specific gene. The most dense arrays may contain tens of thousands of these fragments in an area smaller than a postage stamp, allowing thousands of hybridization reactions to be performed in parallel. Some microarrays are prepared from large DNA fragments that have been generated by PCR and then spotted onto the slides by a robot. Others contain short oligonucleotides that are synthesized on the surface of the glass wafer with techniques similar to those that are used to etch circuits onto computer chips. In either case, the exact sequence—and position—of every probe on the chip is known. Thus, any nucleotide fragment that hybridizes to a probe on the array can be identified as the product of a specific gene simply by detecting the position at which it is bound.

To use a DNA microarray to monitor gene expression, mRNA from the cells being studied is first extracted and converted to cDNA. The cDNA is then labeled with a fluorescent probe. The microarray is incubated with this labeled cDNA sample and hybridization is allowed to occur. The array is then washed to remove cDNA that is not tightly bound, and the positions in the microarray to which labeled DNA fragments have bound are identified by an automated scanning-laser microscope. The array positions are then matched to the particular gene whose sample of DNA was spotted in this location. Typically the fluorescent DNA from the experimental samples (labeled, for example, with a red fluorescent dye) are mixed with a reference sample of cDNA fragments labeled with a differently colored fluorescent dye (green, for example). Thus, if the amount of RNA expressed from a particular gene in the cells of interest is increased relative to that of the reference sample, the resulting spot is red. Conversely, if the gene's expression is decreased relative to the reference sample, the spot is green. If there is no change compared to the reference sample, the spot is yellow. Using such an internal reference, gene expression profiles can be tabulated with great precision. So far, DNA microarrays have been used to examine many changes in gene expression. Indeed, because microarrays allow the simultaneous monitoring of large numbers of genes, they can detect subtle changes in a cell, changes that might not be manifested in its outward appearance or behavior.

Comprehensive studies of gene expression also provide an additional layer of information that is useful for predicting gene function. Information about a gene's function can be deduced by identifying genes that share its expression pattern. Using a technique called cluster analysis, one can identify sets of genes that are coordinately regulated. Genes that are turned on or turned off together under different circumstances are likely to work in concert in the cell: they may encode proteins that are part of the same multiprotein machine, or proteins that are involved in a complex coordinated activity. Characterizing a gene whose function is unknown by grouping it with known genes that share its transcriptional behavior is sometimes called “guilt by association.” Cluster analyses have been used to analyze the gene expression profiles that underlie many interesting biological processes.

In addition to monitoring the level of mRNA corresponding to expression of genes in a genome, DNA microarrays have many other uses. For example, microarrays can be used to quickly identify disease-causing microbes by hybridizing DNA from infected tissues to an array containing genomic DNA sequences from large collections of pathogens. Microarrays can also be used to examine gene expression associated with disease progression.

### Pharmaceutical Manufacturing

Pharmaceutical Solids. The discovery and development of new chemical entities (NCEs) into stable, bioavailable, marketable drug products is a long, but rewarding process. Due to the tremendous cost of developing a NCE, and industry's need to enhance productivity, it is desirable to create NCEs that have suitable physical-chemical properties, rather than compensate for deficiencies solely by the formulation process. Hence, property-based design can enhance the likelihood a NCE will have the desired physical-chemical properties that will facilitate its ability to be developed into a stable, bioavailable dosage form. Even so, well-designed preformulation studies are necessary to fully characterize molecules during the discovery and development process so that NCEs have the appropriate properties, and there is an understanding of the deficiencies that must be overcome by the formulation process.

Once a NCE is selected for development, choosing the molecular form that will be the active pharmaceutical ingredient (API) is the next milestone. Salt selection is the first API decision, in which absorption needs to be balanced with consistency of the API solid state. Excipients are the backbone of a formulation. They may need to stabilize the API. Moreover, judicious choices must be made to prevent incompatibilities between the API and excipients.

A wide variety of different solid states are possible. Polymorphs exist when the drug substance crystallizes in different crystal packing arrangements all of which have the same elemental composition. Hydrates exist when the drug substance incorporates water in the crystal lattice. Desolvated solvates are produced when a solvate is desolvated and the crystal retains the structure of the solvate. Amorphous forms exist when a solid with no long range order and thus no crystallinity is produced.

Solutions, Emulsions, Suspensions, and Extracts. With regard to solutions, emulsions, suspensions, and extracts, the dosage forms are prepared by employing pharmaceutically and therapeutically acceptable vehicles. The active ingredient(s) may be dissolved in aqueous media, organic solvent or combination of the two, by suspending the drug (if it is insoluble) in an appropriate medium, or by incorporating the medicinal agent into one of the phases of an oil and water emulsion. These dosage forms can be formulated for different routes of administration: orally, introduction into body cavities, or external application.

A solution is a homogeneous mixture that is prepared by dissolving a solid, liquid, or gas in another liquid and represents a group of preparations in which the molecules of the solute or dissolved substance are dispersed among those of the solvent. An emulsion is a two-phase system prepared by combining two immiscible liquids, in which small globules of one liquid are dispersed uniformly throughout the other liquid. The word “suspension” is defined as a two-phase system consisting of an undissolved or immiscible material dispersed in a vehicle (solid, liquid, or gas). Extraction, as the term is used pharmaceutically, involves the separation of medicinally active portions of plant or animal tissues from the inactive or inert components by using selective solvents in standard extraction procedures. Absorption occurs when drugs are in a dissolved state, thus it is frequently observed that the bioavailability of oral dosage forms decreases in the following order: aqueous solution>aqueous suspension>tablet or capsule. Formulation may influence the bioavailability and pharmacokinetics of drugs in solution, including drug concentration, volume of liquid administered, pH, ionic strength, buffer capacity, surface tension, specific gravity, viscosity and excipients. Emulsions and suspensions are more complex systems; consequently, the bioavailability and pharmacokinetics of these systems may be affected by additional formulation factors such as surfactants, type of viscosity agent, particle size and particle-size distribution, polymorphism and solubility of drug in the oil phase.

Parenteral Preparations. With respect to parenteral preparations, parenteral dosage forms differ from all other drug dosage forms because they are injected directly into body tissue through the primary protective system of the human body, the skin, and mucous membranes. They must be exceptionally pure and free from physical, chemical, and biological contaminants. These requirements place a heavy responsibility on the pharmaceutical industry to practice current good manufacturing practices (cGMPs) in the manufacture of parenteral dosage forms and upon pharmacists and other health care professionals to practice good aseptic practices (GAPs) in dispensing them for administration to patients. Formulation principles require that parenteral drugs be formulated as solutions, suspensions, emulsions, liposomes, microspheres, nanosystems, and powders to be reconstituted as solutions. Since most liquid injections are quite dilute, the component present in the highest proportion is the vehicle. The vehicle for most parenteral products is water.

The United States Pharmacopeia (USP) requires Water for Injection (WFI). The USP permits substances to be added to a preparation to improve or safeguard its quality, for example, antimicrobial agents, buffers, antioxidants, tonicity agents, and cryoprotectants and lyoprotectants. In the preparation of a parenteral product, the general manufacturing process entails procurement, processing, packaging, and QA/QC. Procurement encompasses selecting and testing of the raw-material ingredients and containers. Processing includes cleaning the equipment, compounding the solution (or other dosage form), filtering the solution, sterilizing the containers, filling measured quantities of product into sterile containers, stoppering, freeze-drying, terminal sterilization, and sealing of the filled container. Packaging constitutes the labeling and cartoning of filled and sealed containers. The quality assurance and control unit is responsible for assuring and controlling the quality of the product through the process.

Ophthalmic Preparations. Ophthalmic preparations are specialized dosage forms designed to be instilled onto the external surface of the eye (topical), administered inside (intraocular) or adjacent (periocular) to the eye. The preparations may have any of several purposes, therapeutic or prophylactic. Topical dosage forms have customarily been restricted to solutions, suspensions, and ointments, but have been expanded to include gels and inserts. The target is usually a tissue of the eye. Ophthalmic use imposes particle size, viscosity, and sterility specifications.

Medicated Topicals. The application of medicinal substances to the skin or various body orifices is a concept taking many forms. Medications are applied to the skin or inserted into body orifices (e.g., rectum, vagina, urethra) in liquid, semisolid, or solid form. Drugs are applied to the skin to elicit an effect on the skin surface, an effect within the stratum corneum, an effect requiring penetration into the epidermis and dermis, or a systemic effect.

Some topical dosage forms are ointments, transdermal drug delivery systems, suppositories, and others. Ointments are semisolid preparations intended for external application to the skin or mucous membranes. The USP recognizes four general classes of ointment bases: hydrocarbon bases, absorption bases, water-removable bases, and water-soluble bases.

Transdermal drug delivery systems, like patches, increase skin residence times from hours to days to permit systemic uptake of the drug or drugs incorporated therein. Suppositories are solid dosage forms for insertion into the rectum, vagina, or urethra. Poultices, pastes, powders, dressings, creams, and plasters are sometimes intended for topical application.

Oral Solid Dosage Forms. Drug substances most frequently are administered orally by means of solid dosage forms such as tablets and capsules. Large-scale production methods used for their preparation require the presence of other materials in addition to the active ingredients. Additives also may be included in the formulations to facilitate handling, enhance the physical appearance, improve stability, and aid in the delivery of the drug to the bloodstream after administration.

These supposedly inert ingredients, as well as the production methods employed, have been shown in many cases to influence the absorption or bioavailability of the drug substances. Therefore, care must be taken in the selection and evaluation of additives and preparation methods to ensure that the drug-delivery goals and therapeutic efficacy of the active ingredient(s) will not be diminished.

Tablets may be defined as solid pharmaceutical dosage forms containing drug substances with or without suitable diluents and have traditionally been prepared by either compression or molding methods. Compressed tablets are formed by compression and, in their simplest form, contain no special coating. They are made from powdered, crystalline, or granular materials, alone or in combination with binders, disintegrants, controlled-release polymers, lubricants, diluents, and in many cases colorants. The vast majority of tablets commercialized today are compressed tablets, either in an uncoated or coated state. In contrast, molded tablets usually are made from moist material, using a mold that gives them the shape of cut sections of a cylinder.

Capsules are solid dosage forms in which the drug substance is enclosed in either a hard or soft, soluble container or shell of a suitable form of gelatin. Among other oral solid dosage forms, pills and lozenges have been replaced largely by compressed tablets, and cachets are related to capsules. Why coat tablets? Sugar coating involves deposition of a coating based on sucrose as a raw material to protect the drug from the environment. Film coating entails deposition of a coating based on polymers, and has all but replaced sugar coating. Film coatings can be applied to tablets, etc. to modify drug release. Enteric coatings are film coatings that generally delay release of a drug. Sustained-release coatings are film coatings that are designed to extend drug release over a period of time. Microencapsulation is a modified form of film coating, differing in the method by which the particles are to be coated. Compression coating involves the use of modified tabletting machines that mediate compaction of a coating around a tablet produced on the same machine.

Controlled Drug Delivery. Controlled drug delivery can be defined as delivery of the drug at a predetermined rate and/or to a location according to the needs of the body and disease states for a definite time period. In rate-controlled release systems, the mechanism by which the release rate is controlled is diffusion, dissolution, osmosis, mechanically driven pump, swelling, erosion, and stimulation. In targeted delivery systems, targeting is achieved by colloidal drug carriers, ligand-mediated targeting, resealed erythrocytes, bioadhesives, and prodrugs. Device implantation, encapsulated cells, and reservoir microchips are being developed. Currently, most modified-release delivery systems fall into the following three categories: Delayed-release, extended-release, and site-specific targeting. Delayedrelease systems are either those that use repetitive, intermittent dosing of a drug from one or more immediate-release units incorporated into a single dosage form, or an enteric delayed release system. Extended-release systems include any dosage form that maintains therapeutic blood or tissue levels of the drug for a prolonged period.

Site-specific targeting refers to targeting a drug directly to a certain biological location. Recently, a novel modification of drug delivery systems has emerged from the pharmaceutical industry, in which a fast-dissolve drug delivery system consists of a solid dosage form that dissolves or disintegrates in the oral cavity without the need of water or chewing. Aerosols. Inhalation therapy has been used for many years, and there has been a resurgence of interest in delivery of drugs by this route of administration. The number of new drug entities delivered by the inhalation route has increased over the past 5 to 10 years. This type of therapy also has been applied to delivery of drugs through the nasal mucosa, as well as through the oral cavity for buccal absorption.

Originally, this type of therapy was used primarily to administer drugs directly to the respiratory system (treatment of asthma); inhalation therapy is now being used for drugs to be delivered to the bloodstream and finally to the desired site of action. Drugs administered via the respiratory system (inhalation therapy) can be delivered either orally or nasally. Further, these products can be developed as a nebulizer/atomizer, dry powder inhaler, nasal inhaler, or metered dose aerosol inhaler.

Routes of Administration. Oral ingestion is the most common method of drug administration. It also is the safest, most convenient, and most economical. Disadvantages to the oral route include limited absorption of some drugs because of their physical characteristics (e.g., water solubility), emesis as a result of irritation to the GI mucosa, destruction of some drugs by digestive enzymes or low gastric pH, irregularities in absorption or propulsion in the presence of food or other drugs, and the need for cooperation on the part of the patient. In addition, drugs in the GI tract may be metabolized by the enzymes of the intestinal flora, mucosa, or liver before they gain access to the general circulation. The parenteral injection of drugs has certain distinct advantages over oral administration. In some instances, parenteral administration is essential for the drug to be delivered in its active form, as in the case of proteins. Availability usually is more rapid, extensive, and predictable when a drug is given by injection. The effective dose therefore can be delivered more accurately. In emergency therapy, parenteral therapy may be a necessity. The injection of drugs, however, has its disadvantages: Asepsis must be maintained.

Injections may be administered by routes such as intravenous, subcutaneous, intradermal, intramuscular, intraarticular, and intrathecal. The type of dosage form (solution, suspension, etc.) will determine the particular route of administration that may be employed. Conversely, the desired route of administration will place requirements on the formulation. For example, suspensions would not be administered directly into the bloodstream because of the danger of insoluble particles blocking capillaries. Solutions to be administered subcutaneously require strict attention to tonicity adjustment. Injections intended for intraocular, intraspinal, intracisternal, and intrathecal administration require stricter standards of such properties as formulation tonicity, component purity, and limit of endotoxins because of the sensitivity of issues encountered to irritant and toxic substances.

Transdermal absorption, pulmonary absorption, topical application to mucous membranes of the conjunctiva, mouth, nasopharynx, oropharynx, vagina, colon, urethra, bladder, and rectum, and ophthalmic application, all are additional routes of administration of primary importance.

Therapeutic Index. The dose of a drug required to produce a specified effect in 50% of the population is the median effective dose, abbreviated ED50. In preclinical studies of drugs, the median lethal dose, as determined in experimental animals, is abbreviated as the LD50. The ratio of the LD50 to the ED50 is an indication of the therapeutic index, which is a statement of how selective the drug is in producing the desired versus its adverse effects.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### Experimental Procedures

The following methods and materials were used in the examples that are described further below.

Cell culture. Human cervical cancer lines CaSki and SiHa were purchased from the American Type Culture Collection and cultured in Dulbecco's Modified Eagle Medium (DMEM) supplemented with 100 IU/mL penicillin, 100 μg/mL streptomycin, and (except where noted) 10% fetal calf serum at 5% CO₂. In several assays, where noted, serum was reduced or removed entirely.

Drugs. LIF (Millipore) was stored in phosphate-buffered saline (PBS) at 4° C. and diluted in DMEM prior to use. TNFa, IL-6 and EGF (Biosource, Invitrogen) were dissolved in PBS with BSA, stored at −20° C., and diluted in DMEM or RPMI prior to use.

Generation of an HPV-16 reporter cell line. The cervical cancer cell line SiHa was chosen because of its relatively high native HPV-16 LCR activity and significant expression of the E6 and E7 genes, similar to that in a progressing dysplastic lesion. The cells were transfected with the plasmid pLCRGLuc, which contains a complete HPV-16 LCR cloned upstream of a secreted Gaussia luciferase gene as well as a neomycin resistance gene driven by an SV40 promoter. Cells were selected with 400 μg/mL G418 for seven days.

Cell proliferation assay. Cell proliferation was determined by CellTiter 96 (Promega) (3-(4,5-Dimethylthiazol-2-yl)-2,5-diphenyltetrazolium bromide (MTT) assay. Cells were seeded at 5,000 per well in a 96-well plate and treated with the specified cytokines. After the indicated periods, the cells were incubated according to the manufacturer's protocol with the MTT labeling solution for 4 h, then with the solubilization/stop solution overnight. Reduction of MTT to MTT-formazan, indicating cellular metabolic activity, was quantified by measurement of absorbance at 570 nm on a SpectraMax microplate reader (Molecular Devices) and background absorbance of 650 nm was subtracted. All experiments were done in triplicate or higher multiplicate for statistical power.

Luciferase assays. For firefly luciferase, cells were seeded in 12-well plates at a density of 50,000/well and transfected with the indicated reporter plasmids using SuperFect (QIAGEN, Alameda Calif.) according to manufacturer's instructions. After 18 hours, medium was replaced and the cells treated with the indicated stimuli for the time periods noted. Luciferase was released from cells using the supplied lysis buffer and luminescence measured using luciferase substrate (New England Biolabs, Ipswich Mass.).

For Gaussia luciferase, SiHa-LCR-gLuc cells were seeded in 96-well plates at a density of ˜5,000/well and treated as indicated. Following incubation, 20 (micro)L of supernatant from each well was transferred to a new plate. Gaussia luciferase reagent (New England Biolabs) was added and the luminescence measured for 5 seconds.

Quantitative real-time PCR. In order to directly measure the expression of E6 and E7 mRNA we designed primer sets and probes specific to the E6 gene and the E7 gene; oligonucleotides were synthesized by Eurofins MWG Operon (Huntsville, Ala.). Primer sequences for the E6 mRNA were 5′-CAAACCGTTGTGTGATTTGTTAATTA-3′ and 5′-GCTTTTTGTCCAGATGTCTTTGC-3′ and the probe was 5′[6-FAM]TGTATTAACTGTCAAAAGCCACTGTGTCCTGAAGAA[TAMRA-6-FAM]-3′, corresponding to nucleotides 382-444. For E7, primer sequences were 5′-GTGACTCTACGCTTCGGTTGT-3′ and 5′GCCCATTAACAGGTGTTCCA-3′ and the probe sequence was 5′[6-FAM]CGTACAAAGCAC.

ACACGTAGACATTCGTAC[BHQ1a-6FAM]-3′, corresponding to nucleotides 743-794 (or 743-1955 in the SiHa variant). Following experimental treatments, RNA was harvested using the RNeasy kit (Qiagen), quantified by spectrophotometry (Nanoprop 8000, Thermo Scientific), equal quantities amplified using the TaqMan kit (Applied Biosystems) according to the standard protocol (qv) on an ABI Prism 7700. We estimated the quantity of HPV mRNA relative to β-actin mRNA using the 2^{-ΔΔc}_{T} method[19].

Intracellular phosphospecific flow cytometry. Trypsinized cells (10,000 for each timepoint) were fixed with 1.2% paraformaldehyde at room temperature for 10 minutes, rinsed with PBS containing 1% BSA, permeabilized in PBS/90% ice-cold methanol, and stored at −20° C. overnight or for up to 2 weeks. Prior to staining, cells were washed twice in PBS containing 1% BSA. Cells were stained on ice with the indicated antibodies for 30 minutes at 4° C. and analyzed on a FC500 flow cytometer (Beckman-Coulter). Further analysis was performed using cytobank (www.cytobank.org).

Statistics Statistical analysis (unpaired t test) was performed using GraphPad software (graphpad.com) as needed. P values indicating statistical significance are represented by a single (P<0.1) or double asterisk (P<0.05) on the figures.

### EXAMPLES

The following example is put forth so as to provide those of ordinary skill in the art with a complete disclosure and description of how to make and use the present invention; it is not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### LIF Inhibits HPV-16 LCR-Driven Transcription

In this study, we compared the transcriptional activity of the HPV-16 LCR in untreated cervical SiHa cells and cells treated with various cytokines. All experiments were performed using the human cervical cancer cell lines CaSki and SiHa. An HPV-16 LCR-driven Gaussia luciferase reporter construct (containing a full LCR fragment from HPV-16 cloned in front of secreted Gaussia luciferase), called pGLucLCR, was constructed and used to transfect SiHa cells which were then selected in G418. These cells were treated with varying concentrations of LIF for 20 hours prior to lysis. Luciferase activity was assayed in triplicate. A STAT3-responsive reporter construct (containing four repeats of the m67, i.e., STAT3-responsive, domain and a minimal tk promoter transcribing luciferase) was used to transiently transfect CaSki cells which were then similarly assayed. We designed a primer and probe set specific to HPV-16 E6, E7 for quantitative RT-PCR. Primer sequences for the E6 mRNA were 5′-CAAACCGTTGTGTGATTTGTTAATTA-3′ and 5′GCTTTTTGTCCAGATGTCTTTGC-3′ and the probe was 5′[6-FAM]TGTATTAACTGTCAAAAGCCACTGTGTCCTGAAGAA[TAMRA-6-FAM]-3′. For E7, primer sequences were 5′-GTGACTCTACGCTTCGGTTGT-3′ and 5′GCCCATTAACAGGTGTTCCA-3′ and the probe sequence was 5′[6-FAM]CGTACAAAGCACACACGTAGACATTCGTAC[BHQ1a-6FAM]-3′. The E6, E7mRNA level for each sample was normalized against that of β-actin. All conditions were tested in triplicate.

Phosphorylated STAT3 (Y705) was detected using a PE-conjugated phosphospecific antibody (BD PhosFlow). We grew CaSki cells to ˜50% confluence in keratinocyte growth medium (Lonza) and replaced the medium with keratinocyte basal medium with 10 ng/mL of LIF, EGF, or IL-6 for 40 hours. Cell growth was determined by MTT assay in sextuplicate

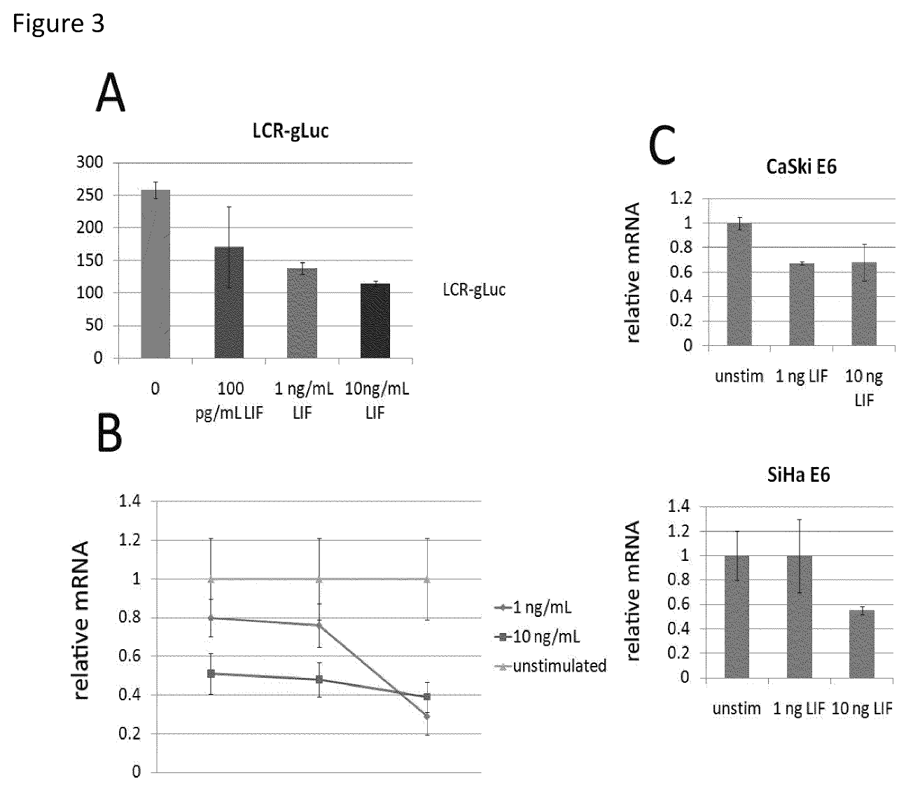

The IL-6 superfamily member LIF reduced HPV-16 LCR expression by approximately 60% in a time- and dose-dependent manner (FIG. 3A). In order to verify that the observed decrease in LCR transcription was functionally significant and not an artifact of the reporter system, we examined the effect of LIF on mRNA expression by quantitative real-time PCR. CaSki cervical cancer cells were treated with 1 ng/mL or 10 ng/mL of LIF or with PBS for one, two, or three days. The expression of E7 mRNA was significantly reduced over this time course, initially in a dose-dependent manner (FIG. 3B), although at three days cells treated with the low or high concentrations of LIF showed similar inhibition of E7; E6 inhibition followed a similar trend (FIG. 3C).

### Example 2

### Induction of Phosphorylation of STAT3 in Cervical Cancer Cells

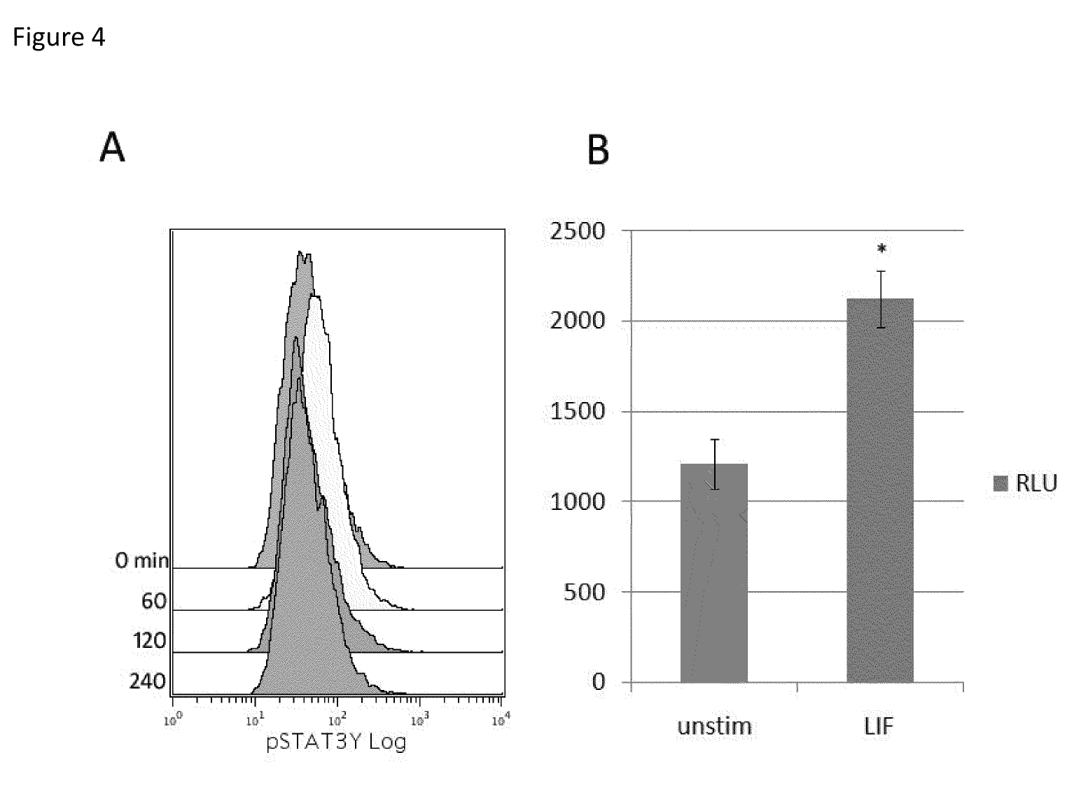

LIF is known to activate members of the JAK-STAT pathway, typically JAK1 and STAT3 in a cell-specific manner through binding to the heterodimeric LIFR-gp130 receptor (Stahl et al., 1994; Megeney et al, 1996) and, therefore, we evaluated the effect of LIF on STAT3 activation in SiHa cells. STAT3 is a multifunctional signaling molecule generally considered to promote survival, proliferation, and tumorigenesis but can also be involved in the initiation of senescence and programmed cell death (Abell et al. 2005). Following treatment with LIF, STAT3 was phosphorylated on tyrosine residue 705, indicative of activation, in a transient fashion (FIG. 4A).

To determine if the observed phosphorylation was accompanied by transcriptional activation, we used the reporter plasmid 4xM67 pTATA TK-Luc; SiHa cells were transfected; after 18 hours cells were treated with 10 ng/mL LIF or PBS. After six hours cells were lysed and analyzed as described. The activity of the reporter plasmid was 1.8-fold higher in LIF-treated cells than in untreated cells (FIG. 4B).

### Example 3

### Proliferation of HPV-Transformed Cells

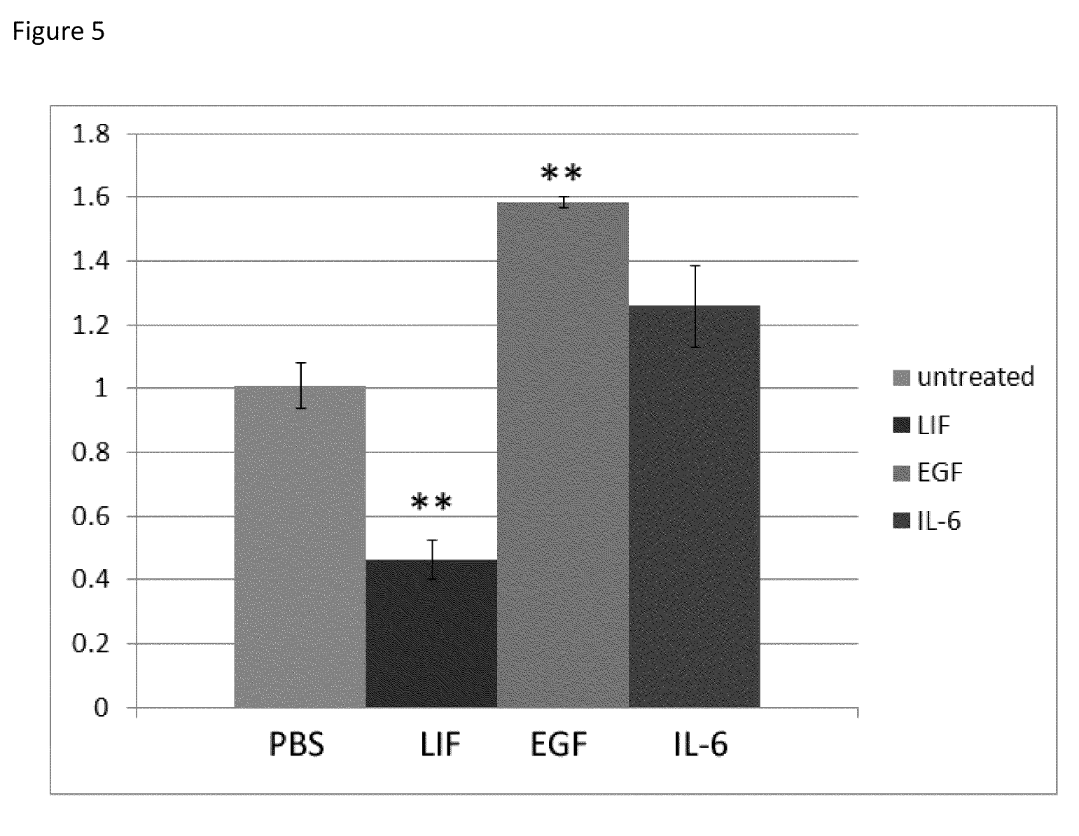

CaSki cells were seeded in 96-well plates at a density of ˜5000/well in growth medium. After the cells attached (˜6 hours) the medium was replaced with serum-free medium containing LIF, the LCR-inhibiting cytokines IL-6 or EGF[22], or PBS. After 40 hours the proliferation of cells was measured using the MTT assay described earlier. As expected, EGF promoted the proliferation of the cells relative to untreated cells. The LIF-treated cells, however, proliferated much less rapidly, only reaching a final population density approximately half that of untreated cells (FIG. 5).

These examples and embodiments are illustrative and are not to be read as limiting the scope of the invention as it is defined by this specification and the appended claims.

### REFERENCES

Abell K et al. (2005). Stat3-induced apoptosis requires a molecular switch in PI(3)K subunit composition. Nat Cell Biol 7: 392-398
De Villiers E M et al. (2004). Classification of Papillomaviruses. Virology 324:17-27.
Doorbar J (2006). Molecular biology of human papillomavirus infection and cervical cancer. Clinical Science 110:525-541.
Dürst M et al. (1992). Human papillomavirus type 16 (HPV 16) gene expression and DNA replication in cervical neoplasia: Analysis by in situ hybridization. Virology 189:132-140.
Goodman A & Wilbur D C (2003). Case 32-2003: A 37-Year-Old Woman with Atypical Squamous Cells on a Papanicolaou Smear. N Engl J Med 349:1555-1564.
Iglesias M et al. (1995). Interleukin-6 and interleukin-6 soluble receptor regulate proliferation of normal, human papillomavirus-immortalized, and carcinoma-derived cervical cells in vitro. Am J Pathol 146: 944-952.
Kyo S et al. (1993). NF-IL6 represses early gene expression of human papillomavirus type 16 through binding to the noncoding region. J Virol 67:1058-1066.
Megeney L A et al. (1996). bFGF and LIF signaling activates STAT3 in proliferating myoblasts. Dev Genet. 19: 139-145.
Stahl N et al. (1994). Association and activation of Jak-Tyk kinases by CNTF-LIF-OSM-IL-6 beta receptor components. Science 263: 92-95.

What is claimed is:

1. A process for treating an HPV-associated papillomatous proliferation in a mammal in need thereof comprising:
administering LIF or polypeptide, said polypeptide being at least 30% identical thereto or with up to 30% insertions, deletions, or conservative substitutions therein, topically to a HPV-associated papillomatous proliferation in a mammal in need thereof.

2. The process of claim 1, wherein said polypeptide comprises an amino acid sequence that is at least 70%, 75%, 80%, 85%, 90%, or 95% identical to SEQ ID NO:1.

3. The process of claim 1, wherein said polypeptide comprises the amino acid sequence of SEQ ID NO:1, with up to 30%, 25%, 20%, 15%, 10%, or 5% insertions, deletions, or conservative substitutions.

4. The process of claim 1, wherein said LIF or polypeptide comprises the amino acid sequence of SEQ ID NO:1.

5. The process of claim 1, wherein said LIF or polypeptide consists of the amino acid sequence of SEQ ID NO:1.

6. The process of claim 1 further comprising as a first step identifying a mammal in need thereof.

7. The process of claim 1, wherein said HPV is HPV-16.

8. The process of claim 1, wherein said HPV is HPV-18.

9. The process of claim 1, wherein said HPV is HPV-31, HPV-33, HPV-35, HPV-45, HPV-52, or HPV-58.

10. The process of claim 1, wherein said LIF or polypeptide is applied topically as an ointment, transdermal drug delivery system, suppository, poultice, paste, powder, dressing, cream, or plaster.

11. The process of claim 1, wherein said administering step is by topical intravaginal application.

12. A process for treating an HPV-associated genital, anal, vulvar, penile, oral, or laryngeal wart in a mammal in need thereof comprising:
administering LIF or polypeptide, said polypeptide being at least 30% identical thereto or with up to 30% insertions, deletions, or conservative substitutions therein, topically to an HPV-associated genital, anal, vulvar, penile, oral, or laryngeal wart in said mammal.

13. The process of claim 12, wherein said polypeptide comprises an amino acid sequence that is at least 70%, 75%, 80%, 85%, 90%, or 95% identical to SEQ ID NO:1.

14. The process of claim 12, wherein said polypeptide comprises the amino acid sequence of SEQ ID NO:1, with up to 30%, 25%, 20%, 15%, 10%, or 5% insertions, deletions, or conservative substitutions.

15. The process of claim 12, wherein said LIF or polypeptide comprises the amino acid sequence of SEQ ID NO:1.

16. The process of claim 12, wherein said LIF or polypeptide consists of the amino acid sequence of SEQ ID NO:1.

17. The process of claim 12 further comprising as a first step identifying a mammal in need thereof.

18. The process of claim 12, wherein said HPV-associated wart is a genital wart.

19. The process of claim 12, wherein said HPV is HPV-16.

20. The process of claim 12, wherein said HPV is HPV-18.

21. The process of claim 12, wherein said HPV is HPV-31, HPV-33, HPV-35, HPV-45, HPV-52, or HPV-58.

22. The process of claim 12, wherein said LIF or polypeptide is applied topically as an ointment, transdermal drug delivery system, suppository, poultice, paste, powder, dressing, cream, or plaster.

23. The process of claim 12, wherein said administering step is by topical intravaginal application.

24. A process for treating HPV-associated cervical dysplasia or cervical cancer in a mammal in need thereof comprising:
administering LIF or polypeptide, said polypeptide being at least 30% identical thereto or with up to 30% insertions, deletions, or conservative substitutions therein, to said mammal.

25. The process of claim 24, wherein said polypeptide comprises an amino acid sequence that is at least 70%, 75%, 80%, 85%, 90%, or 95% identical to SEQ ID NO:1.

26. The process of claim 24, wherein said polypeptide comprises the amino acid sequence of SEQ ID NO:1, with up to 30%, 25%, 20%, 15%, 10%, or 5% insertions, deletions, or conservative substitutions.

27. The process of claim 24, wherein said LIF or polypeptide comprises the amino acid sequence of SEQ ID NO:1.

28. The process of claim 24, wherein said LIF or polypeptide consists of the amino acid sequence of SEQ ID NO:1.

29. The process of claim 24 further comprising as a first step identifying a mammal in need thereof.

30. The process of claim 24, wherein said mammal is diagnosed with HPV-associated cervical dysplasia classified as CIN1, CIN2, or CIN3.

31. The process of claim 24, wherein said HPV is HPV-16.

32. The process of claim 24, wherein said HPV is HPV-18.

33. The process of claim 24, wherein said HPV is HPV-31, HPV-33, HPV-35, HPV-45, HPV-52, or HPV-58.

34. A process for repressing HPV transcription in a mammal in need thereof comprising:
administering LIF or polypeptide, said polypeptide being at least 30% identical thereto or with up to 30% insertions, deletions, or conservative substitutions therein, to said mammal.

35. The process of claim 34, wherein said LIF or polypeptide comprises the amino acid sequence of SEQ ID NO:1.

36. The process of claim 34, wherein said LIF or polypeptide consists of the amino acid sequence of SEQ ID NO:1.

37. The process of claim 34, wherein said HPV is HPV-16.

38. The process of claim 34, wherein said HPV is HPV-18.

39. The process of claim 34, wherein said HPV is HPV-31, HPV-33, HPV-35, HPV-45, HPV-52, or HPV-58.

40. The process of claim 34, wherein said LIF or polypeptide is applied topically as an ointment, transdermal drug delivery system, suppository, poultice, paste, powder, dressing, cream, or plaster.

41. The process of claim 34, wherein said administering step is by topical intravaginal application.

42. A kit comprising:
a. purified or recombinant LIF or polypeptide, said polypeptide being at least 30% identical thereto or with up to 30% insertions, deletions, or conservative substitutions therein, and b. a component configured to collect a cervical swab or biopsy sample to test for HPV.

43. The kit of claim 42, wherein said LIF or polypeptide comprises the amino acid sequence of SEQ ID NO:1.

44. The kit of claim 42, wherein said LIF or polypeptide consists of the amino acid sequence of SEQ ID NO:1.

45. The kit of claim 42, wherein said HPV is HPV-16.

46. The kit of claim 42, wherein said HPV is HPV-18.

47. The kit of claim 42, wherein said HPV is HPV-31, HPV-33, HPV-35, HPV-45, HPV-52, or HPV-58.
